(19) United States
(12) Patent Application Publication
(10) Pub. No.: US 2011/0033842 A1
(43) Pub. Date: Feb. 10, 2011

(54) Skin Sampling Kit Which Stores Nucleic Acids In Stable Status, Genetic Test Methods By Using The Kit And Their Practical Application

(76) Inventors: Woo Chul Moon, Seoul (KR); Jin Yung Lee, Incheon (KR); Keun Yang Park, Gyeonggi-do (KR); Jung Sik Shing, Seoul (KR)

Correspondence Address: HODGSON RUSS LLP;THE GUARANTY BUILDING, 140 PEARL STREET, SUITE 100, BUFFALO, NY, 14202-4040, US

(21) Appl. No.: 12/935,970

(22) PCT Filed: Apr. 4, 2008
(86) PCT No.: PCT/KR08/01917

§ 371 Date: Oct. 1, 2010

(51) Int. Cl. C12Q 1/70; C07H 1/06; C12P 19/34; C12Q 1/68; C12M 1/00; A61B 10/00

(52) U.S. Cl. 435/5; 536/23.5; 435/91.2; 435/6; 435/283.1; 600/562

(57) ABSTRACT

The present invention relates to a new skin gene card for genetic test, a method for acquiring DNA and RNA and performing various genetic tests using the same, and practical applications thereof. More specifically, the inventors of the present invention have developed a skin gene card capable of acquiring samples from human skin, hair or mucosa simply, safely and quickly and enabling stable long-term storage and transport of DNA and RNA included in the acquired sample at room temperature. Various genetic tests may performed using the acquired DNA and RNA, including polymerase chain reaction (PCR), reverse transcription (RT)-PCR, real-time PCR, sequencing, hybridization, DNA chip analysis, single-nucleotide polymorphism (SNP) assay, gene mutation assay, promoter methylation assay, gene expression assay, etc. The genetic skin test result may be utilized for disease prognosis, nutrigenomic test, pharmacogenomic test, forensic test such as personal identification, diagnosis of genetic diseases, diagnosis of skin diseases, or the like. In addition, through an objective evaluation of the skin or hair condition, a personalized cosmetic and skin care system may be established for practical application in beauty care, cosmetology, dermatology, and clinical practice.

### TECHNICAL FIELD

The present invention relates to a new skin gene card for genetic test, a method for acquiring DNA and RNA and performing various genetic tests using the same, and practical applications thereof. More specifically, the inventors of the present invention have developed a skin gene card capable of acquiring samples from human skin, hair or mucosa simply, safely and quickly and enabling stable long-term storage and transport of DNA and RNA included in the acquired sample at room temperature. Various genetic tests may performed using the acquired DNA and RNA, including polymerase chain reaction (PCR), reverse transcription (RT)-PCR, real-time PCR, sequencing, hybridization, DNA chip analysis, single-nucleotide polymorphism (SNP) assay, gene mutation assay, promoter methylation assay, gene expression assay, etc. The genetic skin test result may be utilized for disease prognosis, nutrigenomic test, pharmacogenomic test, forensic test such as personal identification, diagnosis of genetic diseases, diagnosis of skin diseases, or the like. In addition, through an objective evaluation of the skin or hair condition, a personalized cosmetic and skin care system may be established for practical application in beauty care, cosmetology, dermatology, and clinical practice.

### BACKGROUND ART

Multicellular organisms including humans consist of numerous cells. The nuclei of the cells have DNAs where genetic information is stored. The basic unit holding the genetic information is called a gene. The gene is a portion of DNA. All the biological phenomena and functions of a cell are mediated by proteins. The gene is a vast information unit directing and transmitting a series of commands for the synthesis of proteins. Each gene has a specific genetic code required for synthesis of one or more specific protein(s).

DNA has a double helical structure. Each helical strand consists of numerous chemical structure units called bases. There are four types of DNA bases: adenine (A), cytosine (C), guanine (G) and thymine (T). The sequence of these bases, or the base sequence, determines the genetic information. For the genetic information of DNA to be materialized into protein production in the cytoplasm, an intermediate mediator is required, which is known as RNA. The genetic information of DNA is first copied into mRNA. This procedure is called the transcription. Then, the genetic information of mRNA is decoded protein in the cytoplasm by a ribosome with the help of tRNA and rRNA (translation). Three bases specify a single amino acid. The tri-nucleotide units are called codons. The protein produced by the ribosome is prepared into an activated protein through posttranslational modification. When a cell is divided, DNA is replicated and transferred to daughter cells identically. An individual has the same DNAs in all cells. The types, structures and functions of all the cells, as well as physical conditions and development of diseases in an individual, are determined by the kind and amount of proteins expressed in the cells, which in turn is determined depending on which RNA is transcribed to what extent. That is to say, the difference in the kind and amount of genes expressed in each cell makes the difference. Actually, the percentage of genes expressed in the individual cells is only 3-5% (Aressns J, Armstrong M, Gilissen R, Cohen N. The human genome: an introduction. Oncologist. 2001; 6: 100-109).

The full set of genes an organism has is called the genome. In contrast, the set of all genes expressed in an individual (i.e. mRNAs) is called the transcriptome, and the entire complement of expressed proteins is called the proteome. The human genome occupies a total of over 3 billion base pairs, and is reported to contain about 30,000 genes. With the recent completion of the Human Genome Project with a primary goal to determine the base sequence of the entire human genome, remarkable developments are made in diagnosis and treatment of intractable diseases using genes, and the era of the so-called personalized medicine and predictive medicine is opening.

The biological phenomena are determined by (1) genetic information of genomic DNAs, (2) transcription of genes, and (3) expressed proteins. Recently, studies are actively carried out to analyze all these information automatically. To this end, microarrays or biochips are of great help. Genetic studies are also carried out actively in the field of dermatology. For example, there are attempts to study the physiology, pathology and function of the skin using such techniques as cDNA microarray. Also, polymerase chain reaction (PCR) or other techniques are used to diagnose skin infection and detect pathogens. Although it is expected that a better understanding an diagnosis of skin disease may be attained through accurate evaluation of skin condition through genetic tests, there are few practical applications or distinct results. It seems that genetic skin tests may be applied to personalized skin treatment, beauty care and makeup, but there are few reports thereabout (Fuller B R et al. Gene array technology and the search for cosmeceutical actives. In: Cosmoceuticals. Edited by Draelos Z D. Elsevier Saunders, 2005). To put the genetic skin test to practical use, a lot of problems have to be solved. In particular, substantial researches on how to adequately acquire skin samples in a noninvasive manner, how to analyze the genes, how to diagnose skin disease and evaluate skin condition based on the result, and how to practically apply it to personalized skin care. The present invention is directed to solving the problems.

One of the biggest problems in genetic researches using DNA or RNA sample is that the nucleic acids are quickly degraded at room temperature. Especially, RNA is degraded in a few hours by ribonuclease (RNase A) which is secreted from cells during the separation process and abundant in the environment. The inventors of the present invention have developed a method for stably storing RNA and DNA in the form of card or liquid at room temperature over a long period of time, using chitosan, and have patented or filed for a patent thereon. RNA and DNA cards, PCR and reverse transcription (RT)-PCR kits, and microarray chips based thereon are used to store, carry and analyze multiple DNA and RNA samples. In the present invention, the RNA card is used for the development of a kit for skin genetic test.

The skin is the largest organ of the body, with an average area of 1.6 m² and accounting for about 16% of body weight in adults. It is very complex in structures, functions, and physiologies. Recently, with the development in molecular genetics and proteomics, new facts are being found out relating the skin's structure, function, physiology, aging and disease development mechanisms.

The skin protects the body from external stimulations or dangers and accustoms the body to environmental changes, for example, through body temperature regulation. Its other functions are sensation, secretion, excretion, incretion of hormones such as vitamin D and cytokines, immunity, and regeneration. Further, it plays a critical role in beauty care.

The skin is composed of three primary layers, the epidermis, the dermis and the subcutaneous adipose layer (subcutaneous tissue), from outside. The appendages of skin include hair, sebaceous glands, sweat glands (eccrine glands), capillary vessels, or the like.

The epidermis is the thinnest of the three skin layers, but plays an important role of moisturizing and protecting the skin. Further, it prevents loss of moisture and damage of tissues, as well as invasion of pathogens. The main type of cells which make up the epidermis are keratinocytes, with melanocytes, Langerhans cells and Merkel cells also present. The keratinocytes move upward from the stratum basale as they are differentiated, thereby forming the outermost horny layer (stratum corneum). Dead keratinocytes are sloughed off at the skin surface. The stratum corneum is the first barrier defending the skin. The melanocytes have long dendrites extending among the keratinocytes. Melanin is shipped to the keratinocytes and absorbs or disperses UV, thereby protecting the skin from damage.

In the aspect of functions, the skin may be seen as a barrier providing protection from harmful materials or stimulations from outside. It is very important to understand this skin's barrier mechanism, as well as physiologies and pathologies. What is the most important in the skin barrier function is the stratum corneum of the epidermis. The stratum corneum is composed of corneocytes and a lipid structure. The stratum corneum is composed proteins (40%), water (40%) and lipids (10-20%). To assimilate the skin layer to a brick wall, the corneocytes are like bricks and the lipid structure serves as plaster.

Skin moisturization is a prerequisite for a healthy skin. It is primarily attained by the stratum corneum. The stratum corneum keeps the skin moisturized by way of (1) natural moisturizing factor (NMF) produced by corneocytes, (2) the lipid layer between the corneocytes, (3) desmosomes, and (4) sebum secreted from the sebaceous gland. The lipid of the epidermis mainly consists of ceramide, cholesterol, and free fatty acid.

The dermis is about 15-40 times thicker than the epidermis and takes up the most volume of the skin. It is composed of two layers, the papillary dermis and reticular dermis. Structural components of the dermis are cells, connective tissues and extracellular matrix. The cells present in the dermis include fibroblasts, histiocytes, mast cells, Langerhans cells, lymphocytes and plasma cells. Besides, there exist (skin appendages such as blood vessels, lymphatic vessels, nerves, arrector pili, eccrine glands, apocrine sweat glands, eccrine ducts, pilosebaceous units, nails, etc. The dermis supplies nutrients to the epidermis, supports the epidermis, protects the body from skin damage, regenerates the skin by cooperating with the epidermis, stores moisture, regulates body temperature, and serves as receptor of sensation.

The connective tissue of the dermis is abundant in fibers such as collagen fiber, elastic fiber, reticular fiber, etc. The major components are collagen and elastin. Particularly, collagen is abundant. There are five types of collagen in the skin types 1, 3, 4, 7 and 8. Among them, type 1 is the most abundant, with 80-85%. Collagen and elastin form a fibrous connective tissue beneath the epidermis, thereby supporting the epidermis and providing elasticity and flexibility. There are enzymes that break down collagen, the most important one among them being matrix metalloproteinase 1 (MMP1, collagenase 1). There also exists a substance in the tissue that inhibits MMP1. It is called tissue inhibitor of metalloproteinase (TIMP). The content of collagen in the skin is maintained constant by collagen synthase, MMP and TIMP. However, when the balance is broken due to decreased collagen synthesis, excessive action of MMP1, decreased TIMP, or the like, the skin loses elasticity and wrinkles are formed because of decreased collagen. Besides the natural aging process, such bad factors as UV, inflammation and superoxide groups accelerates MMP1 generation, thereby accelerating skin aging and worsening wrinkles.

The dermal matrix is composed of glycosaminoglycans or mucopolysaccharides. The chief components are hyaluronic acid (also called hyaluronan) and chondroitin sulfate. Heparan sulfate is also included. These substances have a very powerful moisturizing ability. There are several types of hyaluronan synthases (HAS) in the skin. Among them, HAS3 exists in the epidermal corneocytes and HAS2 exists in the dermal fibroblasts. Recently, it was observed that the water channel protein aquaporin 3 (AQP3) is expressed in the skin. The protein may play an important role in regulating skin moisturization.

The subcutaneous tissue, also called the subcutaneous adipose layer, is composed of adipose tissue. It supplies nutrients to the epidermis and the dermis, determines the body shape, maintains the body temperature, and serves as thermal insulator of the body. It lies below the dermis, consists of blood vessels, lymphatic vessels, nerves and adipose cells, and functions as a cushion to resist pressure from outside.

The human skin is commonly classified into 4 types, depending on the contents of sebum and moisture: (1) normal type, (2) oily type, (3) dry type, and (4) mixed type. Recently, a sensitive type is added as the fifth skin type, and the degree of aging is evaluated along with the skin type. However, the skin type may change incessantly because it is affected by various factors including age, sex, hormonal state, nutritional state, life pattern, environment, and the like. The classification of skin type is very important for adequate skin care and selection of cosmetics.

Skin care and cosmetics are among the most important things with regard to the skin. The definition of cosmetics is slightly different from one country to another. In Korea and Japan, cosmetics are defined as substances anointed, sprayed or otherwise applied to the skin or hair to keep the human body clean, beautiful or healthy, and with little action on the human body. In contrast, in the US, cosmetics are defined as substances used to clean, beautify or enhance the appearance of the human body without structural or functional change. In Europe, teeth or oral mucosa are also included. In contrast, the substances having pharmaceutical effects or structurally or functionally changing the human body are classified as drugs. Recently, a lot of cosmetic products belonging somewhere between the two, with effects on the structure or function of human skin, are produced. They are called cosmeceuticals (Sung-ku Ahn, Seung-Hun Lee. Skin aesthetics. Korea Medical Book Publisher. 2002; Cosmeceuticals. Edited by Draelos Z D. Elsevier Saunders, 2005).

Basically, cosmetics have to be stable, safe, effective and pleasant. Here, the effectiveness refers to the effect in physicochemical, physiological and psychological aspects. For example, it refers to moisturizing, anti-wrinkling, anti-aging, skin-whitening, softening, coloring or cleansing effect. Since the skin type and condition are different from person to person, it is important to select suitable cosmetics. Further, it is important to establish standards by which the effect before and after the use of cosmetics can be accurately and objectively evaluated (Sung-ku Ahn, Seung-Hun Lee. Skin aesthetics. Korea Medical Book Publisher. 2002).

Test methods used to evaluate the human skin condition and the effect of cosmetics or cosmeceuticals include: (1) morphological test (imaging study), (2) skin color analysis, (3) skin softness and elasticity test, (4) skin temperature and blood flow test, (5) transepidermal water loss (TEWL) test, (6) skin hydration test, (7) lipid content evaluation, (8) UV blocking effect test, (9) hair moisturization and damage evaluation, and (10) ultrasonic test (Sung-ku Ahn, Seung-Hun Lee. Skin aesthetics. Korea Medical Book Publisher. 2002; Grove G L et al. Evaluating cosmeceutical efficiency. In: Cosmeceuticals. Edited by Draelos Z D. Elsevier Saunders, 2005).

However, most of these tests focus one of skin structure, shape, physiology or pathology, and are limited for actual application because of lack of objectivity and reproducibility. Accordingly, a new test method capable of accurately and objectively evaluating the human skin condition, thereby being of help in classifying the skin type, selecting personalized cosmetics or cosmeceuticals, and evaluating the effect after application thereof. The present invention is also directed thereto.

Today, the traditional concept of cosmetics of beautifying and cleaning the human body and keeping the skin or hair healthy is changing with the advent of functional cosmetics for actively changing and improving the skin. That is, the cosmeceuticals functioning both as cosmetics and pharmaceuticals is becoming the mainstream. The cosmetics industry is a comprehensive industry encompassing basic and applied techniques of chemistry, biology, pharmacology and dermatology. Recently, as molecular genetics is introduced thereto, attempts are made to understand the skin's physiological activities and molecular pathologies more accurately and to develop personalized skin care, cosmetics and cosmeceuticals.

A variety of diseases develop on the skin, with various symptoms and signs. The skin diseases include genetic diseases, psychocutaneous disorders, photosensitive skin diseases, skin diseases induced by physical factors, occupational skin diseases, urticaria, erythema, drug eruption, eczema, psoriasis, immune disorders, infections, sexually transmitted diseases, pigmentary disorders, vascular diseases, connective tissue disorders, subcutaneous tissue disorders, sebaceous gland and sweat gland diseases, hair diseases, nail diseases, benign and malignant tumors, precancerous lesions, and mucosal diseases. It is not uncommon that skin diseases are caused by systemic diseases such as endocrinopathy or metabolism disorder. Infections may be caused by bacteria, tubercle bacilli, fungi, viruses, parasites, or the like. Sexually transmitted infections may cause skin diseases, too.

The symptoms occurring in the skin include itching (pruritus), scorching, burning, pain, hypoesthesia, anesthesia, etc. The skin disease-related signs include the original primary lesion and the secondary lesion which develops from the primary lesion. The primary lesions include macule, patch, papule, plaque, nodule, tumor, wheal, vesicle, etc., and the secondary lesions include scale, crust, excoriation, erosion, ulcer, scar, fissure and lichenification.

The diagnosis of the skin appears easy because it can be seen directly. However, different skin diseases may exhibit similar symptoms and signs, and different aspects may be observed for the same patient and for the same disease, depending on the stages. Accordingly, the diagnosis may be difficult only with subjective examination of symptoms by interview or physical examinations of the signs. Even the dermatologists find it difficult to diagnose some diseases. Tests for the diagnosis of skin disease include Gram staining and culturing for detecting bacterial infection, KOH staining and culturing for detecting fungal infection, the Tzanck test for detecting herpes simplex and herpes zoster, scabies scraping for detecting scabies, dark-field examination for detecting syphilis, patch test, stimulating the skin by injection, pricking or scratching and monitoring the response, dermographism test, diascopic examination, Wood's lamp examination, and the like. Unless a diagnosis is made through the above tests, skin biopsy, in which a skin tissue is observed under an optical microscope after staining, by immunohistochemical staining or immunofluorescence test, or using an electronic microscope, may be necessary (Sung-ku Ahn, Seung-Hun Lee. Skin aesthetics. Korea Medical Book Publisher. 2002; Korean Dermatological Association Textbook Publishing Committee. Dermatology. 4th Edition. Ryo Moon Gak. 2001).

However, all the aforesaid tests merely microscopically monitor the structural change of skin lesion, and fail to monitor the physiological, functional, biochemical, molecular and genetic changes thereof. Therefore, they are limited in accuracy and effectiveness. Accordingly, there is an urgent need of a new test method capable of identifying the fundamental cause and development of skin diseases and determining optimally personalized therapies.

The condition and type of skin are determined by the genes expressed in the skin, changes in the composition of proteins, carbohydrates, lipids, etc. produced thereby, and the status of the cells constituting the skin. Not only inherited genetic factors, but also acquired factors such as environmental factors, diets and life patterns affect them. Thus, investigation of inherited genetic factors and examination of the genes expressed in the skin will provide the most accurate and fundamental knowledge of the skin condition. The present invention is also directed thereto.

### DISCLOSURE

### Technical Problem

At present, medical examinations by interview, physical examinations, physical and chemical examinations, and morphological examinations using various instruments are carried out for the evaluation of skin condition and diagnosis in skin disease in dermatological clinics, cosmetic and plastic clinics, beauty care shops and cosmetics companies worldwide. However, the existing test methods are restricted in fundamentally and objectively evaluating all the individuals skins. Further, there is no scientifically standardized test method as yet. The most objective test method available now is one monitoring the change of microstructure of the skin following biopsy. However, this method is invasive, and the physiological, functional or biochemical changes cannot be monitored. Therefore, a new test method capable of accurately and objectively evaluating the human skin condition, thereby being of help in classifying the skin type, selecting personalized cosmetics or cosmeceuticals, and evaluating the effect after application thereof. An object of the present invention is to provide such a method.

In this respect, the most promising method is genetic test. The condition and type of skin and the onset of skin disease are determined by the genes expressed in the skin, changes in the composition of proteins, carbohydrates, lipids, etc. produced thereby, and the status of the cells constituting the skin. Not only inherited genetic factors, but also acquired factors such as environmental factors, diets and life patterns affect them. Thus, investigation of inherited genetic factors and examination of the genes expressed in the skin will provide the most accurate and fundamental knowledge of the skin condition. However, a lot of problems remain to be solved for the skin genes to be practically applied. First, a method for safely acquiring skin sample appropriate for test and for transporting the same is not established. Second, a method for acquiring specific genes from the acquired skin sample and for performing various genetic tests including polymorphism, mutation and expression is not established. Third, a method and a standard for utilizing the test result for actual clinical diagnoses or cosmetics purposes are not established. If it can be acquired safely and simply, the skin may be the best sample for various genetic tests.

### Technical Solution

As described above, the human skin is composed of several layers. In the dermis and the epidermis, even in the same epidermis, different cells i.e. keratinocytes, melanocytes, Langerhans cells, etc. express different genes. Accordingly, a standardization or normalization ensuring stable skin sampling and with uniform thickness will be a prerequisite. Besides, for a skin sampling method applicable not only for clinical purposes but also for cosmetics or other purposes, the method needs to be safe, noninvasive and simple. If possible, a “do it your self (DIY)” method that can be used by the public is preferred. To overcome the shortcomings of the existing genetic skin test methods, a method enabling safe and sure skin sampling is required. Besides, it is to be ensured that DNA and RNA may be acquired from the skin sample with good quality and proper quantity.

Further, complicated and various genetic tests should be possible with the DNA and RNA included in the sample acquired using a skin gene card. Examples of such tests are as follows: polymerase chain reaction (PCR) for amplifying some or all of DNAs of a specific gene, reverse transcription (RT)-PCR for amplifying some or all of DNAs a specific expressed gene, real-time RT-PCR for quantifying expression level of a gene, cloning of a gene acquired by PCR and RT-PCR using a plasmid vector and E. coli, restriction fragment length polymorphism (RFLP) analysis following PCR, base sequencing of a specific gene by way of automated sequencing analysis or oligonucleotide microarray (oligo DNA chip) followed by analysis of single-nucleotide polymorphism and mutation, simultaneous analysis of difference in gene expression by way of cDNA microarray, and analysis of promoter methylation by way of methylation specific PCR (MSP) and bisulfite genomic sequencing.

Thirdly, the established genetic test method should be applicable to actual clinical practices, beauty care and other fields. For example, the skin type needs to be classified more accurately and objectively through accurate evaluation of the skin's functions of body protection, moisturization, regeneration, etc., so that the result may be utilized for selecting personalized skin care, cosmetics and cosmeceuticals. Particularly, the test method should be of help in determining the dry, aged, photoaged or sensitive skin and treating them. Besides, it should be possible to accurately diagnose intractable skin diseases including inflammation, eczema, immune-related disease, infection, psoriasis, etc. and select an adequate therapy. Further, the test method should be applicable to a variety of genetic tests, including diagnosis of hereditary genetic disease, personal identification and paternity testing, genotyping prior to organ transplantation, or the like.

The present invention is directed to providing solutions to these problems.

### ADVANTAGEOUS EFFECTS

A skin gene card kit according to the present invention enables noninvasive and simple sampling of various samples from the skin, hair, mucosa, etc. of the human body and enables storage and transport of the sample with the DNA and RNA included in the sample being safe for a long period of time even at room temperature. DNA and RNA may be easily and stably acquired from the sample, and they may be applied to various genetic tests including polymerase chain reaction (PCR), reverse transcription (RT)-PCR, real-time PCR, PCR-restriction fragment length polymorphism (RFLP), northern hybridization, cloning, base sequencing, oligonucleotide microarray analysis, methylation specific PCR (MSP), bisulfite genome sequencing, or the like. Thus, it may be applied to single-nucleotide polymorphism (SNP) assay, mutation analysis, gene expression assay, etc. The skin gene card kit and a genetic test method established by the present invention may be utilized to more accurately evaluate the skin condition by examining the expression of 30 genes playing a critical role in the functions, physiologies and pathologies of the skin and to classify the skin type more accurately and objectively. Further, the result may be of help in selecting personalized skin care, cosmetics and cosmeceuticals. Particularly, in the field of beauty care and cosmetics, it will be of help to diagnose dry, aged or sensitive skin and care and treat them. Using the skin gene card kit and genetic test method established by the present invention, a variety of skin diseases including tumor, inflammation, eczema, immune-related disease, infection, etc. may be more accurately diagnosed and a personalized therapy may be selected for individual skin diseases. In addition, the skin gene card and genetic test method of the present invention may be utilized for various genetic tests including simple and safe diagnosis of hereditary genetic disease, personal identification and paternity testing, genotyping prior to organ transplantation, or the like.

### DESCRIPTION OF DRAWINGS

The above and other aspects, features and advantages of the disclosed exemplary embodiments will be more apparent from the following detailed description taken in conjunction with the accompanying drawings in which:

FIG. 1 shows a flow sheet of the present invention;

FIG. 2 shows a skin gene card prepared by attaching a paper bandage tape (3M) to an RNA card (Goodgene Corporation) as an embodiment of the present invention;

FIG. 3 shows a test result about whether DNA can be acquired from the skin gene card (lane 1: negative control, lane 2: sample acquired from the skin gene card after a day of storage, lane 3: sample acquired from the skin gene card after 3 days of storage, lane 4: sample acquired from the skin gene card after 7 days of storage);

FIG. 4 shows a polymerase chain reaction (PCR) analysis result about whether DNA remains without degradation in skin cells acquired using the skin gene card after long-term storage at room temperature and whether it can be amplified and analyzed (lane 1: sample acquired from the skin gene card after 5 days of storage, lane 2: sample acquired from the skin gene card after 15 days of storage, lane 3: sample acquired from the skin gene card after 30 days of storage);

FIG. 5 sows an RNA separation result using EasySpin kit (Intron) from a skin sample acquired using the skin gene card (lane 1: 1 Kbp size marker, lane 2: 1 ug sample, lane 3: 0.5 ug sample);

FIG. 6 shows a reverse transcription (RT)-PCR result of the β-actin gene using RNA acquired 1 day, 1 week and 1 month after sampling using the kit in order to investigate whether RNA remains without degradation in skin cells acquired using the skin gene card after long-term storage at room temperature (lane 1: 100 bp DNA marker, lane 2: negative control, lane 3: sample stored for a day, lane 4: sample stored for a week, lane 5: sample stored for a month);

FIG. 7 shows a test result about whether DNA can be separated from the skin gene card after hair is sampled with the root attached (lane 1: 40 Kbp T7 DNA, lane 2: sample stored for a day, lane 3: sample stored for a month, lane 4: sample stored for a year);

FIG. 8 shows a test result about whether RNA can be separated from the skin gene card after hair is sampled with the root attached (lane 1: RNA marker, lane 2: sample stored for a day, lane 3: sample stored for a month, lane 4: sample stored for a year);

FIG. 9 shows a test result about whether PCR of a specific gene is possible without separation of DNA from the skin gene card (lane M: 100 bp marker, lane 1: negative control, lane 2: positive control (HaCaT cell line), lane 3: skin sample from normal adult);

FIG. 10 shows a test result about whether RT-PCR of a specific gene is possible without separation of RNA from the skin gene card (lane M: 100 bp marker, lane 1: positive control (HaCaT cell line), lane 2: skin sample from normal adult);

FIG. 11 shows a test result about whether real-time PCR can be performed using RNA separated from the skin gene card (lane 2: β-actin at 300 ng, lane 3: β-actin at 2000 ng, lane 4: β-actin at 30,000 ng, lane M: 100 bp DNA marker, lane 5: sample for MMP1 gene, lane 6: sample for COL1A1 gene, lane 7: sample for elastin gene, lane 8: sample for elastase gene, lane 9: sample for TIMP gene, lane 10: sample for elafin gene);

FIG. 12 shows a gene amplification result for cloning the gene acquired from the skin gene card (lane 1: 100 bp DNA marker, lane 2: MMP1 gene product).

FIG. 13 shows a map of a vector for cloning the gene amplified through PCR;

FIG. 14 shows a sequencing result after cloning MMP1 gene into pGEM-T Easy vector;

FIG. 15 shows a result of extracting skin genomic DNA using the skin gene card and performing PCR for single-nucleotide polymorphism (SNP) analysis of cardiovascular disease-related genes followed by electrophoresis of the product on 1.5% agarose gel (lanes 1 and 13: 100 bp DNA size marker, lanes 2 and 3: eNOS gene, lanes 4 and 5: MTHFR gene, lanes 6 and 7: AGT gene, lane 8: ACE gene, lanes 9 and 10: AT1R gene, lanes 11 and 12: ApoE gene);

FIG. 16 shows a result of extracting skin genomic DNA using the skin gene card and performing PCR for SNP analysis of cardiovascular disease-related genes followed by electrophoresis of the product on 1.5% agarose gel after treating with restriction enzymes given in Table 3 (lanes 1, 12 and 13: 100 bp DNA size marker, lanes 2 and 3: eNOS gene, lanes 4 and 5: AGT gene, lanes 6 and 7: ACE gene, lane 8: AT1R gene, lanes 9 and 10: ApoE gene, lanes 11 and 14: MTHFR gene);

FIG. 17 shows a result of extracting skin genomic DNA using the skin gene card and performing PCR of p53 tumor suppressor gene which plays an important role in carcinogenesis followed by electrophoresis of the product on 1.5% agarose gel (lane 1: 100 bp DNA size marker, lane 2: negative control, lane 3: positive control, lanes 4 and 6: test sample);

FIG. 18 shows a result of performing electrophoresis of the PCR product of FIG. 17 on 1.5% agarose gel, isolating and purifying the product and analyzing base sequence using ABI 3130 sequencer for identification of mutation of p53 tumor suppressor gene (175 C→A);

FIG. 19 shows a result of genotyping of RNA sample by way of oligonucleotide microarray after acquiring squamous cell carcinoma skin sample using the skin gene card and storing (Mutation of p53 gene (exon 7, codon 282 CGG→TGG) of squamous cell carcinoma patient was identified using CanScan DNA chip (Goodgene));

FIG. 20 shows a result of northern blotting test for identifying expression of a specific gene using RNA acquired from the skin gene card (lane M: RNA marker, lane 1: sample 1 from normal adult, lane 2: sample 2 from normal adult, lane 3: sample 3 from normal adult, lane 4: sample 4 from normal adult);

FIG. 21 schematically shows that methylation only at the cytosine residue at 5′-position of CpG dinucleotide of DNA;

FIG. 22 shows a result of testing the occurrence of methylation at a specific gene from the DNA acquired from the skin gene card (lane 1: 100 bp DNA marker, lane 2: negative control, lane 3: sample acquired from the skin gene card);

FIG. 23 shows a chemical modification procedure for confirming methylation of C base at specific portion of a gene using DNA sample acquired from the skin gene card (When the DNA sample is treated with sodium bisulfite, the unmethylated cytosine base of the CpG island in the base sequence is replaced by uracil (thymine));

FIG. 24 shows a result of performing PCR of MYOD gene using the sodium bisulfite-treated DNA sample of FIG. 23 followed by electrophoresis of the product on 1.5% agarose gel (lane 1: 100 bp DNA size marker, lane 2: MYOD gene PCR product);

FIG. 25 shows a result of base sequencing of the MYOD PCR product of FIG. 24 using ABI 3130 sequencer for identifying whether DNA was accurately methylated by sodium bisulfite treatment in FIG. 23 (As indicated by the arrows, the unmethylated cytosine base of the CpG island was replaced by uracil (thymine));

FIG. 26 shows a result of performing multiplex-PCR of 9 short tandem repeat (STR) loci (D3S1358, D5S818, D7S820, D8S1179, D13S317, D18S51, D21S11, FGA, and vWA) using AmpF1 STR Profiler Plus PCR amplification kit (Applied Biosystems) followed by electrophoresis on 1.5% agarose gel, for personal identification (paternity testing) of skin genomic DNA extracted using the skin gene card (lane 1: 500 bp DNA size marker, lanes 2 and 4: card sample, lane 5: negative control, lane 6: 100 bp DNA size marker);

FIG. 27 shows a result of performing PCR of two VNTR loci (D1S80, D17S30) followed by electrophoresis on 1.5% agarose gel, for personal identification (paternity testing) of skin genomic DNA extracted using the skin gene card (lanes 1-5: D1S80; lane 1: 100 bp DNA size marker, lanes 2 and 4: card sample, lane 5: negative control, lanes 6-10: D17S30; lane 6: 100 bp DNA size marker, lanes 7, 8 and 9: card sample, lane 10: negative control);

FIG. 28 shows a result of analysis of the PCR product of FIGS. 26 and 27 using ABI 3130 genetic analyzer (Applied Biosystems) and GeneMapper ID program (Human Identification Detection, Applied Biosystems) (A: STR marker D3S1358 internal control size marker, B and C: standard for measurement of STR marker D3S1358 PCR product from card sample);

FIG. 29 shows a result of performing PCR of CYP2D6 gene, a representative drug-metabolizing gene, and investigating CYP2D6 polymorphism by PCR-restriction fragment length polymorphism (RFLP) and sequencing for skin genomic DNA extracted using the skin gene card;

FIG. 30 shows a result of CYP2D6 allele frequency calculation based on the result of FIG. 29;

FIG. 31 shows a result of performing single- and multiplex-PCR of representative genes followed by electrophoresis on 1.5% agarose gel for skin genomic DNA extracted using the skin gene card (Single-PCR was performed using TNF-αgene; lane M: 100 bp DNA size marker, lanes 9 and 10: card sample, lane Conventional: DNA extracted from blood. Multiplex-PCR was performed using 5 genes (COMT, CYP1A1-1, CYP1B1, IL-6 and VDR); lane M: 100 by DNA size marker, lanes 9 and 10: card sample, lane Conventional: DNA extracted from blood.);

FIG. 32 shows a result of base sequencing using ABI 3130 sequencer after electrophoresis of the PCR product of FIG. 31 on 1.5% agarose gel and isolation and purification of the product to confirm that the PCR product of TNF-αgene is not false positive;

FIG. 33 shows a result of analysis using ABI 3130 Genetic analyzer (GeneMapper program) following multiplex-PCR as described in FIG. 31 and treatment with SNaPshot Multiplex kit (Applied Biosystems) for identification of SNP, for skin genomic DNA extracted using the skin gene card;

FIG. 34 shows a result of performing multiplex-PCR of 18 nutrigenomic genes (genes involved in obesity, antioxidative stress, detoxification, cardiovascular disease, hormone metabolism, allergy and bone metabolism) followed by imaging analysis using AW (Anti-aging and Well being) chip (Goodgene) for skin genomic DNA extracted using the skin gene card (The result shows that −3826 A of the hormone metabolism-related CYP1A1 gene is replaced by G.);

FIG. 35 shows a result of performing PCR of APC gene, one of the genes causing genetic diseases, followed by electrophoresis of the product on 1.5% agarose gel, for skin genomic DNA extracted using the skin gene card (lane 1: 100 bp DNA marker, lane 2: APC PCR product from card sample);

FIG. 36 shows a result of base sequencing for mutation of the APC gene (1493 G→A) using ABI 3130 sequencer after performing electrophoresis of the APC PCR product of FIG. 35 on 1.5% agarose gel followed by isolation and purification of the product;

FIG. 37 shows a result of acquiring skin sample using the skin gene card as in Example 5, synthesizing cDNA therefrom, performing PCR using a primer specific to the skin cancer-related gene MAGE, and electrophoresis of the product on 1.5% agarose gel (lanes 1 and 9: 100 bp DNA marker, lanes 2 and 3: negative control, lane 4: positive control, lanes 5 and 8: card sample);

FIG. 38 shows a result of separating DNA from the skin gene card and analyzing staphylococcal infection using Staphylococcus aureus PCR kit (Goodgene) (Analysis result of the PCR product using an automated base sequencer revealed infection by Staphylococcus aureus);

FIG. 39 shows a result of separating DNA from a sample (perianal) acquired using the skin gene card and detecting sexually transmitted disease using 12 STD Multiplex PCR kit (Goodgene) (The result reveals infection by HPV. lane 1: 100 bp size marker, lane 2: PCR product from sample, lane 3: STD B set positive control);

FIG. 40 shows a result of imaging analysis of the HPV positive PCR product from the sample (perianal) acquired in Example 26 using the skin gene card, using GG HPV genotyping chip (Goodgene) (The result reveals infection by HPV type 11.);

FIG. 41 shows a result of performing nested PCR of DNA acquired from a skin sample with wart-like patch using the skin gene card by a conventional Mycobacterium tuberculosis technique (lane 1: 100 bp DNA size marker, lane 2: negative control, lane 3: positive control, lane 4: PCR product from sample acquired from patient);

FIG. 42 shows a result of performing real-time PCR for MMP1 gene, one of the genes involved in skin condition and health, using the sample acquired using the skin gene card;

FIG. 43 shows a result of performing real-time PCR for AQP3 gene, one of the genes involved in skin condition and health, using the sample acquired using the skin gene card;

FIG. 44 shows a result of performing real-time PCR for Has3 gene, one of the genes involved in skin condition and health, using the sample acquired using the skin gene card;

FIG. 45 shows a result of performing real-time PCR for tyrosinase, one of the genes involved in skin condition and health, using the sample acquired using the skin gene card;

FIG. 46 shows a result of performing real-time PCR for TRP1 gene, one of the genes involved in skin condition and health, using the sample acquired using the skin gene card; and

FIG. 47 shows the expression profile of MMP1 gene created as a database for ages.

### BEST MODE

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second” and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The present invention relates to a kit (hereinafter referred to as a skin gene card) capable of acquiring, transporting and examining human skin sample under an optimal condition so that the genes included therein can be adequately conserved, a method for performing genetic test using the same, and a method for applying the same in various fields such as medicine, beauty care, cosmetology, genetics, and the like.

The inventors of the present invention have noticed that individuals skin condition can be exactly understood by adequately acquiring skin sample and examining expression or mutation of key genes related with the skin's functions, physiologies and pathologies. Thus, they aimed at establishing a method for acquiring skin sample under an optimal condition enabling such genetic tests. To do so,

1) A skin gene card was designed based on the RNA card and DNA card patented by the inventors of the present invention, and a preparation method thereof was established.

2) An optimal skin sampling method using the card was determined.

3) Conditions for storing and transporting the skin sample were established.

4) A method for adequately separating DNA and RNA from the acquired skin sample and a cDNA synthesis condition were established.

5) Methods for genetic tests such as polymerase chain reaction (PCR), reverse transcription (RT)-PCR, real-time PCR, PCR-restriction fragment length polymorphism (RFLP), automated base sequencing, DNA microarray, methylation specific PCR (MSP), etc. of important skin-related genes were established.

6) A system was established so that skin type can be accurately and objectively classified through examination of genes using the skin gene card and the test methods of 5) and personalized skin care, cosmetics and cosmeceuticals can be selected based on the result. Particularly, focus was made on detecting and treating dry, aged, photoaged and sensitive skin.

7) A system was established so that a variety of intractable skin diseases such as inflammation, eczema, immune-related disease, infection, psoriasis, etc. can be diagnosed using the skin gene card and the test methods of 5) and an optimal treatment can be selected.

8) A system was established so that the skin gene card and the test methods of 5) can be used for detection of hereditary genetic diseases and various genetic tests including personal identification, paternity testing, genotyping prior to organ transplantation, or the like.

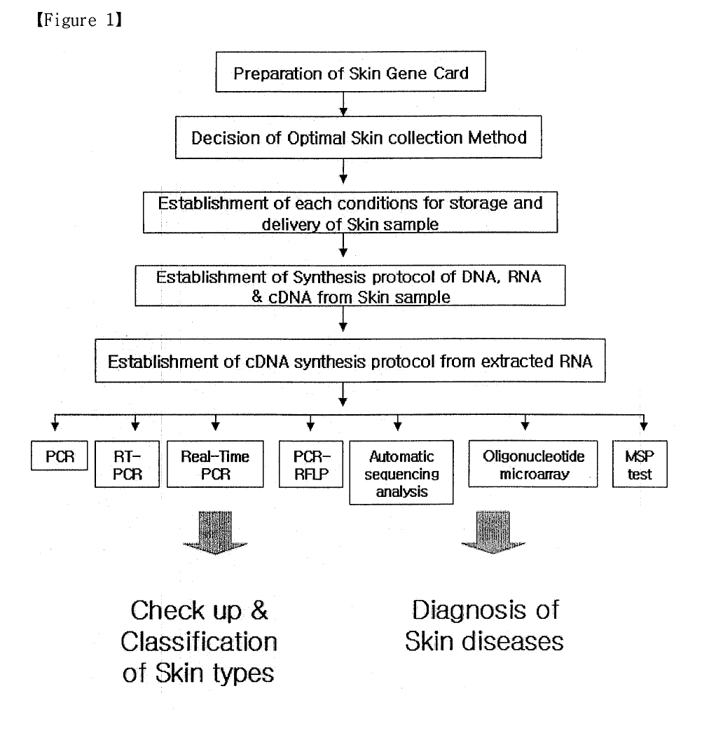

These are summarized in FIG. 1.

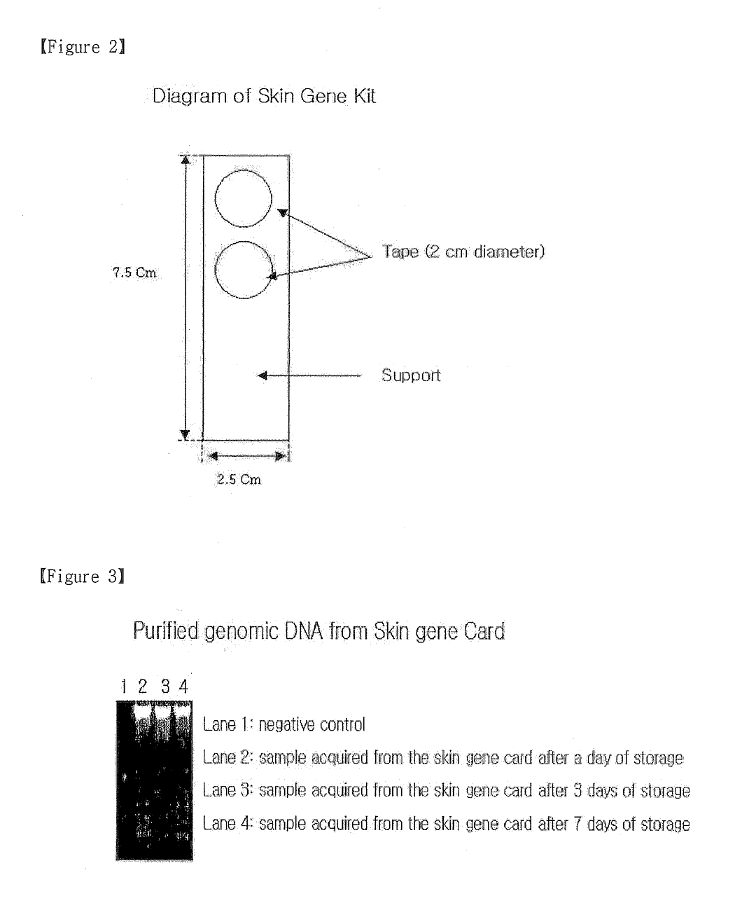

A schematic view of a skin gene card according to the present invention is shown in FIG. 2. The skin gene card of the present invention comprises a tape portion and a card (substrate) portion. The tape is used to acquire tissue from the human body by attaching and detaching it to and from the human body, and the card portion is used to protect, store and transport the tape. The tape may be any kind of adhesive tape. In an embodiment, a bandage tape unharmful to the human body and allowed for medical use, particularly soft, low-tack paper bandage, may be used. Especially, 3M's low-tack type paper bandage tape 1500, 1522 or 9874 may be used. The substrate (card) portion may be a paper card or film, glass slide, plastic, fiber or synthetic resin treated with diethylpyrocarbonate (DEPC), which forms a stable compound with DNA and RNA, in order to prevent the DNA and RNA from being degraded by deoxyribonuclease and ribonuclease, respectively, and to store them stably at room temperature. Further, the substrate portion may be immersed in a lysis buffer or a water-soluble chitosan solution with adequate form and concentration.

Preferably, the human body sample may be human skin. The skin sample may be taken from any portion of the body. Further, the human body sample may be hair or mucosa taken at the skin-mucosa interface, such around the mouth or anus, or inside the mouth.

Whilst the human skin sample used for the genetic test according to the present invention may be acquired using the skin gene card of the present invention, any other of gel- or tape-type apparatus or card for acquiring a small quantity of skin sample may be used for the genetic test according to the present invention.

The target substance component in the sample may be any one that can be indicative of genes, including DNA. Separation of DNA from the skin sample may be performed using an elution buffer. However, any kind of method may be used for the purpose.

More preferably, the target substance component in the sample may be RNA. Separation of RNA and mRNA from the skin sample may be performed using an elution buffer. However, any kind of elution method may be used for the purpose.

[Mode for Invention]

The examples and experiments will now be described. The following examples and experiments are for illustrative purposes only and not intended to limit the scope of this disclosure. Modifications and applications of the examples are included in the scope of the present invention.

<Step 1>

Preparation of Skin Gene Card for Acquiring Skin Sample and Verification of Performance Thereof

In this step, a skin gene card was prepared and a preparation method thereof was established. Further, a method for acquiring an optimal skin sample using the card and conditions for storing and transporting the sample were established. Further, conditions for separating DNA and RNA from the skin sample and for synthesis of cDNA were established. In addition, quality and quantity of the separated DNA and RNA were verified.

Details are as follows.

### Example 1

### Preparation of Skin Gene Card

The skin gene card of the present invention comprises a tape portion and a substrate (card) portion. The tape is used to acquire tissue from the human body by attaching and detaching it to and from the human body, and the card portion is used to protect, store and transport the tape. The tape may be any kind of adhesive tape. In an embodiment, a bandage tape unharmful to the human body and allowed for medical use, particularly soft, low-tack paper bandage, may be used. Especially, 3M's low-tack type paper bandage tape 1500, 1522 or 9874 may be used. The substrate (card) portion may be a paper card or film, glass slide, plastic, fiber or synthetic resin treated with diethylpyrocarbonate (DEPC), which forms a stable compound with DNA and RNA, in order to prevent the DNA and RNA from being degraded by deoxyribonuclease and ribonuclease, respectively, and to store them stably at room temperature. Further, the substrate portion may be immersed in a lysis buffer or a water-soluble chitosan solution with adequate form and concentration. The card was immersed in DEPC treated H20, chitosan and lysis buffer for 30 min, at 120° C. and 2 atm using an autoclave and sterilized and dried before use. This is to prevent contamination of DNA and RNA by DNase and RNase during the separation. And, chitosan, lysis buffer, and DNA and RNA cards provide protection of the nucleic acids for a long time (FIG. 2).

The following table shows the composition and materials of the skin gene card.

| Substrate (card) | Tape |
|---|---|
| RNA card, DNA card, OHP film, | Low-tack tape 1509, |
| slide glass, polyester plastic | 1522, 9874 (3M) |

### Example 2

### Method of Acquiring Skin Sample Using Skin Gene Card

Peeling gel is applied on and around the sampling site. The horny substance is removed by rubbing with hands and the peeling gel is cleanly removed with alcohol. The skin gene card of the present invention is attached on the sampling site with the cover of the tape portion removed. After a while, the card is detached. In an embodiment, acnepris (Biolee) may be used for the peeling gel. However, any gel used for skin cleansing may be used. The duration of time during which the card is attached to the skin may be from 1 minute to 12 hours, commonly 30 minutes. The card of the present invention may be attached on any skin portion. For the purpose of beauty care for those with no skin disease, the sampling is normally performed from forehead, nose, chin, eye rims, or cheek. For the purpose of diagnosis of those who are suspected of skin disease, the sampling may be performed directly at the lesion portion. In this case, it is important to take sample also from the normal portion for comparison.

### Example 3

### Separation of DNA from Skin Gene Card and Identification Thereof

A method for separating DNA from the skin gene card was determined considering the separation of DNA from a trace amount of skin cells and the prevention of interruption of enzymatic reactions e.g. PCR by elution of the substance included in the card or other factors. The DNA separation may also be performed using a variety of commercialized DNA separation kits. However, it may be performed as described below according to the known method using a common extraction buffer. Following the DNA separation, the separated DNA sample was subjected to electrophoresis on agarose gel and UV spectrophotometry. Details are as follows.

Skin samples were acquired from the face of normal adults using the skin gene card and were stored for a day, 3 days and a week, respectively. The total genomic DNA was separated from each card according to the known method (Sambrook J and Russell D W. Molecular cloning: a laboratory manual. Cold Spring Harbor Press. 2001:7.1.-7.88). Triple distilled water was used.

1) The sample is transferred to a 1.5 mL tube and loaded in a microcentrifuge. After adding 1×PBS (500 μL, centrifuge is performed at 12,000 rpm for 2 minutes so that the cells are sedimented.

2) The cells are mixed well with the solution under vortex.

3) Centrifuge is performed at 12,000 rpm for 2 minutes and the supernatant is removed.

4) Buffer TL (200 μL) is added.

5) After adding protease K (20 μL), the mixture is mixed well under vortex.

6) The mixture is incubated at 56° C. for 30 minutes.

7) After completion of reaction, the tube is spun down at 8,000 rpm or above for about 10 seconds so that the solution adhering to the lid is dropped.

8) Buffer TB (400 μL) is added and mixed well. The tube is spun down at 8,000 rpm or above for about 10 seconds so that the solution adhering to the lid is dropped.

9) A spin column is equipped at a collection tube, and the above reaction solution is added to the spin column.

10) Centrifuge is performed at 8,000 rpm for 1 minute.

11) The filtrate passing through the column is discarded and another collection tube is mounted.

12) After adding buffer BW (700 μL), centrifuge is performed at 8,000 rpm for 1 minute.

13) The filtrate passing through the column is discarded and another collection tube is mounted.

14) After adding buffer NW (500 μL), centrifuge is performed at 12,000 rpm for 3 minutes.

15) The filtrate passing through the column is discarded and a fresh 1.5 mL tube is mounted.

16) After adding buffer AE (200 μL) or purified water at the middle portion of the column, the tube is left for 2 minutes at room temperature.

17) Centrifuge is performed at 8,000 rpm for 1 minute.

18) The extracted genomic DNA is subjected to PCR immediately or stored at −20° C. for later use.

19) The extracted genomic DNA is subjected to electrophoresis on 0.8% agarose gel at 100 V and examined under UV.

20) Distilled water (200 μL) is added to a fresh 1.5 mL microcentrifuge tube and left at room temperature for 1 minute. Centrifuge is performed at 8,000 rpm for 1 minute to elute DNA. The separated DNA is subjected to spectrophotometry for concentration measurement, and A260/A280 is compared to determine the purity of the separated DNA. The A260/280 value is between 1.6 and 1.8. As a result, 1-5 μg (average 3 μg) of pure DNA could be acquired from the skin area of 1×2 cm using the skin gene card [FIG. 3].

### Example 4

### Verification of Long-Term Storage of DNA Using Skin Gene Card

It was verified through PCR whether DNA remains without degradation in skin cells acquired using the skin gene card after long-term storage at room temperature and whether it can be amplified and analyzed.

Skin samples were acquired from the face of normal adults using the skin gene card and were stored for a day, a month and a year, respectively. The total genomic DNA was separated from each card. PCR was performed as follows to verify whether the target genes are adequately amplified.

As a result, β-actin gene was distinctly detected in all the DNA samples that had been stored at room temperature for a day, a month and a year.

This result verifies that genomic DNA can be stably stored for at least a year using the skin gene card of the present invention and the stored DNA can be subjected to PCR analysis without any problem. The present invention provides stable storage of DNA similarly to the existing ultra-low temperature storage. The storage temperature may vary from room temperature to −70° C. A dry, dark area is suitable for the storage.

PCR

After a day, a month or a year of storage, Gapdh gene is subjected to PCR using the nucleic acid extracted from the skin gene card as template under the following general conditions (45 cycles).

1) The template (7 ul) and H20 (6 ul) are mixed with PCR mix (10 pM forward and reverse primers each 1 ul, 10× reaction buffer 2 ul, 5 mM dNTP 2 ul, 50 U/ul Taq polymerase 1 ul) to prepare a reaction solution.

2) Reaction is carried out for 45 cycles with 95° C./10 min, 94° C./1 min, 55° C./1 min, 72° C./1 min.

3) Upon completion of the reaction, the tube is spun down at 8,000 rpm or above for about 10 seconds so that the solution adhering to the lid is dropped.

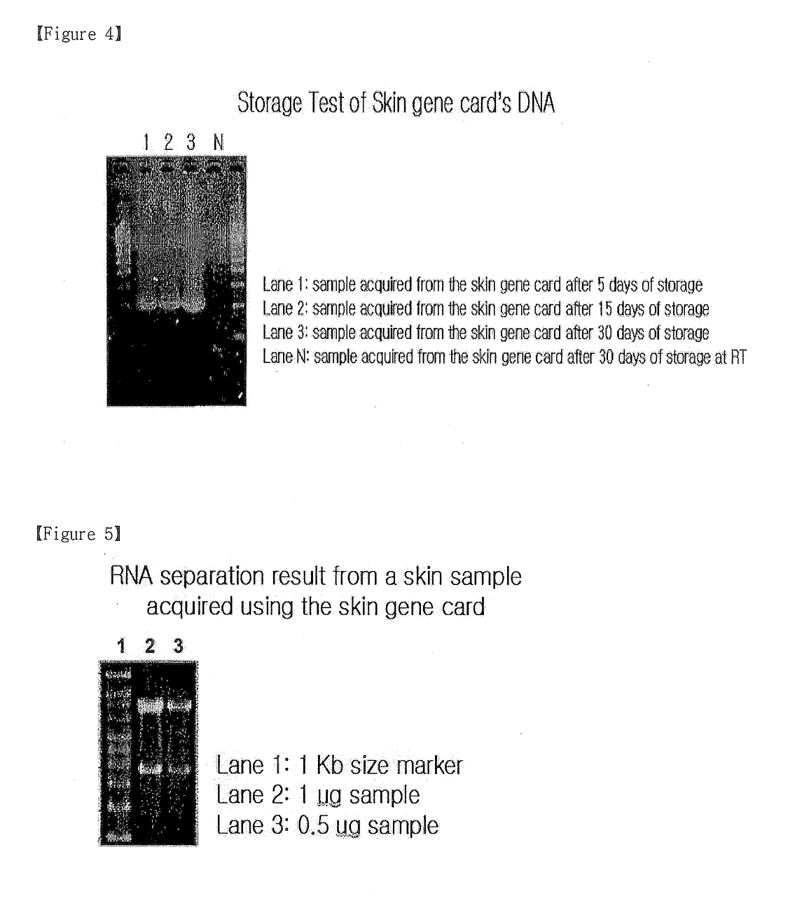

4) The PCR product is subjected to electrophoresis on 0.8% agarose gel at 100 V and examined under UV. An example of the result is shown in FIG. 4.

### Example 5

### Verification of Separation of RNA from Skin Gene Card

RNA was separated from the skin sample acquired using the skin gene card according to a general method. A commercialized EasySpin kit (Cat #17221, Intron) may be used instead. UV spectrophotometry of the separated RNA sample revealed that 5-10 ng/ul of RNA was obtained for a total of 50 ul. OD260/280 was between 1.5 and 1.8. That is to say, 250-500 ng, (average 400 ng) of pure RNA could be acquired from the skin area of 1×2 cm using the skin gene card [FIG. 5].

RNA Separation

1) The sample is transferred to a 1.5 mL tube. After adding lysis buffer (200 μL), the sample and the solution are mixed well for 2 minutes under vortex.

2) Chloroform (200 μL) is added thereto to remove lipid and the sample and the solution are mixed well for 30 seconds under vortex.

3) After centrifuge at 4° C. and 12,000 rpm for 5 minutes, the supernatant is transferred to a fresh tube (Caution is required to prevent the subnatant from being entailed).

Follow procedures 4)-9) when using the EasySpin kit (Intron) else go to 10).

4) Binding buffer (400 μL) is added to the separated supernatant.

5) The solution is loaded on a column and, after keeping at room temperature for 1 minute, centrifuge is performed at 13,000 rpm for 30 seconds.

6) Washing buffer A (700 μL) is added to the column and centrifuge is performed at 13,000 rpm for 30 seconds.

7) Washing buffer B (700 μL) is added to the column and centrifuge is performed at 13,000 rpm for 30 seconds.

8) Centrifuge is performed again at 4° C. and 13,000 rpm for 3 minutes to completely remove water.

9) Elution buffer (50 μL) is added and, after keeping at room temperature for 1 minute, centrifuge is performed at 4° C. and 13,000 rpm for 3 minutes to acquire RNA.

10) After adding isopropanol (same volume with the supernatant of 3)), the mixture is stored at −70° C. for 1-2 hours.

11) The sample is centrifuged at 4° C. and 13,000 rpm for 30 minutes so that RNA is sedimented and the supernatant is discarded.

12) The sedimented RNA is dried using a vacuum dryer and dissolved in pure distilled water (50 ul).

13) The extracted total RNA is subjected electrophoresis on 1.8% agarose gel containing formaldehyde at 100 V and examined under UV.

### Example 6

### Verification of RNA State after Long-Term Storage Using Skin Gene Card

The problem in long-term storage of nucleic acid sample at room temperature is that RNA may be degraded by ribonuclease which is very stable and can be found anywhere in the earth. It was verified through reverse transcription (RT)-PCR analysis whether RNA remains without degradation in skin cells acquired using the skin gene card after long-term storage at room temperature and whether it can be amplified and analyzed.

Skin samples were acquired from the face of normal adults using the skin gene card and were stored for a day, a week and a month, respectively. RNA was separated from each card and RT-PCR was performed as follows to verify whether the target genes are adequately amplified.

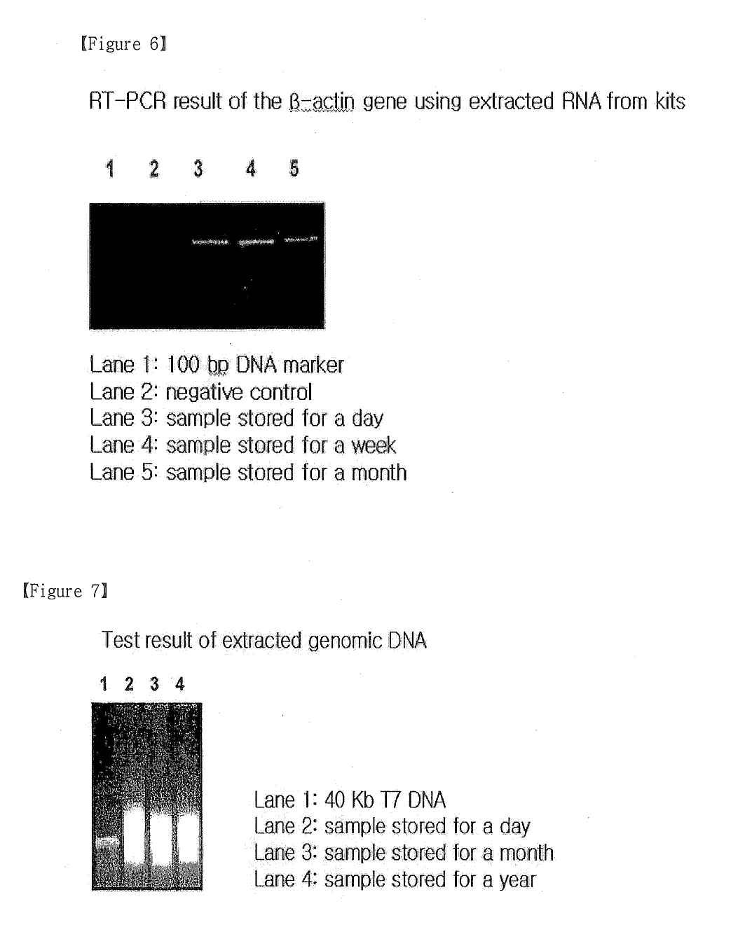

As a result, β-actin gene was distinctly detected in all the skin samples that had been stored at room temperature for a day, a week and a month [FIG. 6].

This result verifies that RNA can be stably stored for at least a month using the skin gene card of the present invention and the stored RNA can be subjected to RT-PCR analysis without any problem. The present invention provides stable storage of RNA similarly to the existing ultra-low temperature storage. The storage temperature may vary from room temperature to −70° C. A dry, dark area is suitable for the storage.

RT-PCR

After a day, a week or a month of storage using the skin gene card, RT-PCR is performed using the extracted RNA as template under the following general conditions.

1) The RNA template (13 ul) is mixed with RT mix (40 ng/ul Oligo-dT 1 ul, 5× reaction buffer 4 ul, 10 mM dNTP 2 ul, 10 U/ul reverse transcriptase 1 ul, RNase inhibitor 1 ul) to prepare a reaction solution.

2) The solution is incubated at 50° C. for 1 hour.

3) Upon completion of the reaction, the tube is spun down at 8,000 rpm or above for about 10 seconds so that the solution adhering to the lid is dropped.

4) The template (13 ul) is mixed with PCR mix (10 pM forward and reverse primers each 1 ul, 10× reaction buffer 2 ul, 5 mM dNTP 2 ul, 50 U/ul Taq polymerase 1 ul) to prepare a reaction solution.

3) Reaction is carried out for 45 cycles with predenaturation (95° C., 10 min) followed by denaturation (94° C., 1 min), annealing (55° C., 1 min) and reaction (72° C., 1 min).

4) Upon completion of the reaction, the tube is spun down at 8,000 rpm or above for about 10 seconds so that the solution adhering to the lid is dropped.

5) The PCR product is subjected electrophoresis on 1.8% agarose gel containing formaldehyde at 100 V and examined under UV.

### Example 7

### Acquiring of Hair Sample Using Skin Gene Card and Separation of DNA Therefrom

Five strands of hair were taken from the human scalp using forceps with the root attached and DNA was separated in the same manner as Example 3 after storing for a day, a month and a year using the skin gene card. The separated DNA sample was subjected to UV spectrophotometry [FIG. 7]. The A260/280 value was between 1.5 and 1.8. As a result, 3-5 μg (average 4 μg) of pure DNA could be acquired from the hair sample.

### Example 8

### Acquiring of Hair Sample Using Skin Gene Card and Separation of RNA Therefrom

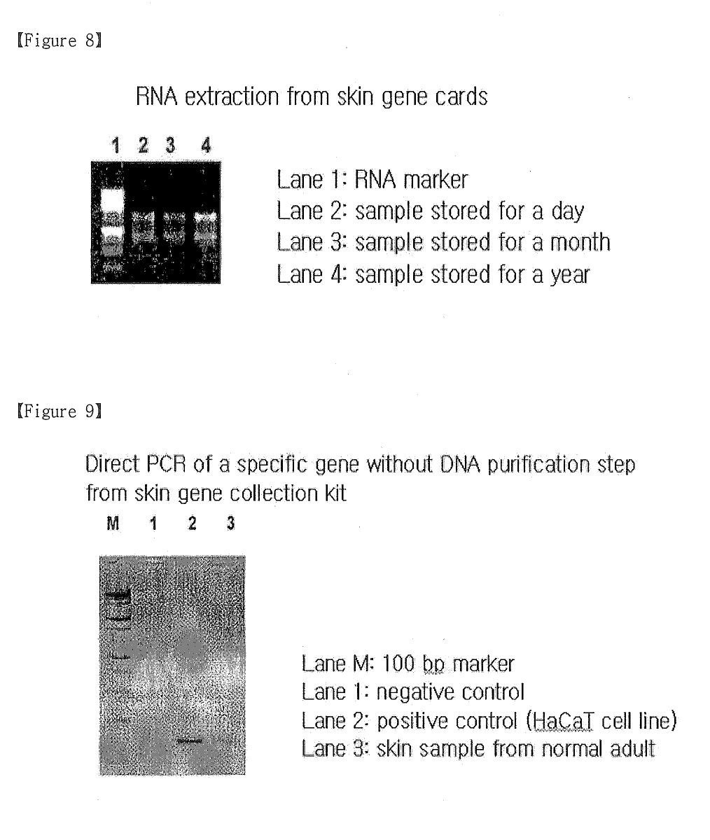

Five strands of hair were taken from the human scalp using forceps with the root attached and RNA was separated in the same manner as Example 5 after storing using the skin gene card as in Example 7. The separated RNA sample was subjected to electrophoresis on 2% agarose gel at 100 V [FIG. 8]. The separated RNA sample was subjected to UV spectrophotometry. The A260/280 value was between 1.5 and 1.8. As a result, 1-2 μg (average 1.5 μg) of pure RNA could be acquired from the hair sample.

<Step 2>

Genetic Tests for Skin Sample Acquired Using Skin Gene Card

In this step, methods for major genetic tests using DNA and RNA included in the sample acquired using the skin gene card were established. First, methods for performing PCR and RT-PCR without separating DNA or RNA from the skin gene card were established. Then, methods for real-time PCR, PCR-RFLP, automated base sequencing, oligonucleotide microarray, cDNA microarray, methylation specific PCR (MSP), bisulfite genome sequencing, etc. were established.

### Example 9

### PCR without Separation of DNA from Skin Gene Card

When performing RT-PCR without separation of DNA from the skin gene card, the prevention of interruption of enzymatic reactions e.g. PCR by RNA or other substances following cell lysis has to be considered in addition to the requirements described in Example 4. In this example, the primer was controlled to make the size of the PCR product of genomic DNA and target RNA different, so that the gene amplification may occur only in the desired genomic DNA.

Skin samples were acquired from the face of normal adults using the skin gene card. Genomic DNA was separated from each card as follows and it was verified whether the target genes are adequately amplified by PCR [FIG. 9].

1) The sample is transferred to a 1.5 mL tube and, after adding Tris-EDTA (pH 7.0) buffer (200 μL), the cells are detached from the tape by vortexing for 5 minutes.

2) After storing the sample at −70° C. for 5 minutes, the cell wall is ruptured by melting in a 60° C. heating block for 1 minute.

3) After centrifuging at 4° C. and 12,000 rpm for 1 minute, the supernatant is transferred to a fresh tube.

4) The template (7 ul) and H20 (6 ul) are mixed with PCR mix (10 pM forward and reverse primers each 1 ul, 10× reaction buffer 2 ul, 5 mM dNTP 2 ul, 50 U/ul Taq polymerase 1 ul) to prepare a reaction solution.

5) Reaction is carried out for 45 cycles with predenaturation (95° C., 10 min) followed by denaturation (94° C., 1 min), annealing (55° C., 1 min) and reaction (72° C., 1 min).

6) Upon completion of the reaction, the tube is spun down at 8,000 rpm or above for about 10 seconds so that the solution adhering to the lid is dropped.

7) The PCR product is subjected to electrophoresis on 0.8% agarose gel at 100 V and examined under UV.

### Example 10

### RT-PCR without Separation of RNA from Skin Gene Card

When performing RT-PCR without separation of RNA from the skin gene card, the prevention of interruption of enzymatic reactions e.g. PCR by genomic DNA or other substances following cell lysis has to be considered in addition to the separation of a trace amount of RNA from skin cells. In this example, the primer was controlled to make the size of the PCR product of genomic DNA and target RNA different, so that the gene amplification may occur only in the desired RNA.

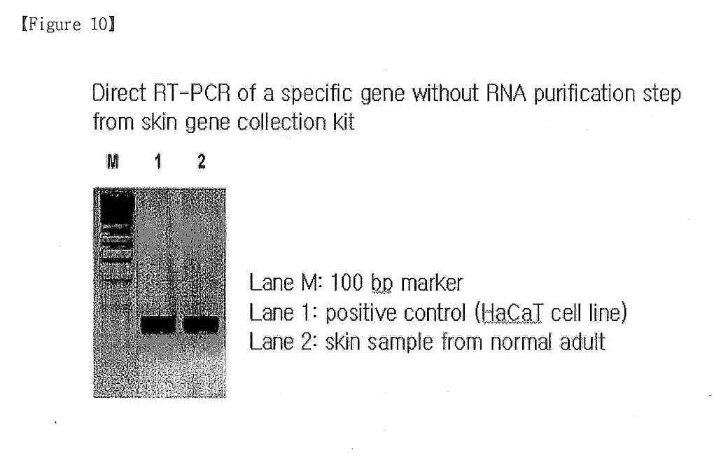

Skin samples were acquired from the face of normal adults using the skin gene card. RNA was separated from each card as follows and it was verified whether the target genes are adequately amplified by RT-PCR [FIG. 10].

1) The sample is transferred to a 1.5 mL tube and, after adding Tris-EDTA (pH 7.0) buffer (200 μL), the cells are detached from the tape by vortexing for 5 minutes.

2) After storing the sample at −70° C. for 5 minutes, the cell wall is ruptured by melting in a 60° C. heating block for 1 minute.

3) After centrifuging at 4° C. and 12,000 rpm for 1 minute, the supernatant is transferred to a fresh tube.

4) The RNA template (13 ul) is mixed with RT mix (40 ng/ul Oligo-dT 1 ul, 5× reaction buffer 4 ul, 10 mM dNTP 2 ul, 10 U/ul reverse transcriptase 1 ul, RNase inhibitor 1 ul) to prepare a reaction solution.

5) The solution is incubated at 50° C. for 1 hour.

6) Upon completion of the reaction, the tube is spun down at 8,000 rpm or above for about 10 seconds so that the solution adhering to the lid is dropped.

7) The template (13 ul) is mixed with PCR mix (10 pM forward and reverse primers each 1 ul, 10× reaction buffer 2 ul, 5 mM dNTP 2 ul, 50 U/ul Taq polymerase 1 ul) to prepare a reaction solution.

8) Reaction is carried out for 45 cycles with predenaturation (95° C., 10 min) followed by denaturation (94° C., 1 min), annealing (55° C., 1 min) and reaction (72° C., 1 min).

9) Upon completion of the reaction, the tube is spun down at 8,000 rpm or above for about 10 seconds so that the solution adhering to the lid is dropped.

10) The PCR product is subjected electrophoresis on 1.8% agarose gel containing formaldehyde at 100 V and examined under UV.

### Example 11

### Real-Time PCR Using Skin Gene Card

Real-time PCR could be performed as follows after separating RNA from the skin gene card.

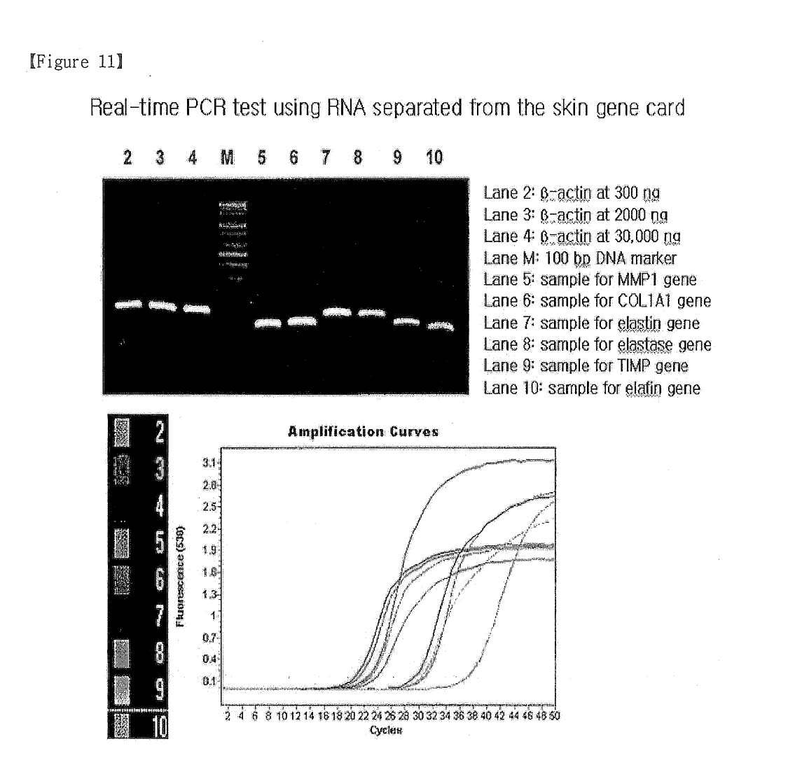

Skin samples were acquired from the face of 20 normal adults, 3 from each person, using the skin gene card. RNA was separated from each card as in Example 5 and it was verified whether the target genes are adequately amplified by one-step real-time PCR [FIG. 11].

1) The Light Cycler reaction condition is set as follows:

Reverse transcription: 50° C., 20 min.

Predenaturation: 94° C., 5 min.

Amplification: 94° C., 15 sec/55° C., 20 sec/72° C., 20 sec.

Melt curve analysis: 95° C., 5 sec/64° C., 15 sec/95° C., 0 sec.

Cooling: 40° C., 30 sec.

2) Reaction solutions for real-time PCR are mixed as follows (number of reactions: 3):

β-Actin (total 20 ul):

Control DNA template 1 μL, 3 μL, 5 μL

DEPC H20 7.8 μL, 5.8 μL, 3.8 μL

Primer 1 ul

Reaction solution 10.2 ul (reaction solution: cyber green mix 185 ul+RT mix 3.7 μL)

Sample (total 20 μL):

Cyber Green mix 10 μL

RT mix 0.2 μL

Primer 1 μL

Template 1, 3, 5 μL

DEPC H20 7.8, 5.8, 3.8 μL

3) The reaction solution is added to a capillary and the lid is covered.

4) Quick spin down is performed on table top centrifuge.

5) The capillary is mounted on the Light Cycler and a run is started.

### Example 12

### Cloning of Genes Acquired from Skin Gene Card

Cloning was performed as follows in order to stabilize the gene products obtained by PCR and RT-PCR in Examples 9 and 10.

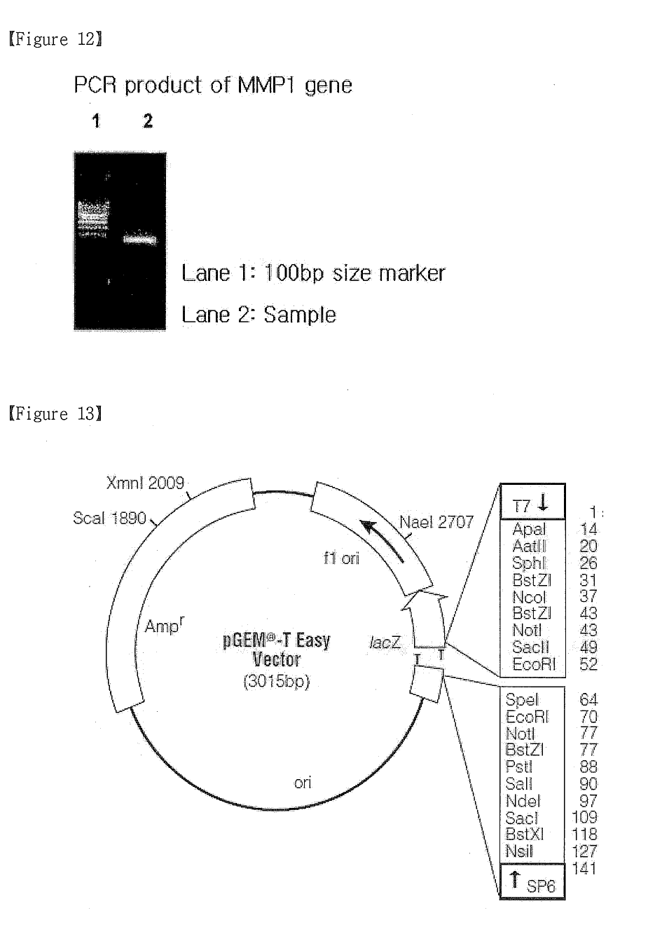

To take MMP1 as an example, PCR was performed first for the sample acquired from the skin gene card in accordance with the present invention. To this end, primers (5′-CCGGTTTTTCAAAGGGAATAA-3′ and 5′-CACAGTTCTAGGGAAGCCAAAG-3′) were prepared and PCR was formed with 30 cycles of 95° C./5 min followed by 95° C./30 sec, 55° C./30 sec and 72° C./30 sec. The PCR product [FIG. 12] was purified and cloned by ligating into pGEM-T Easy vector [FIG. 13].

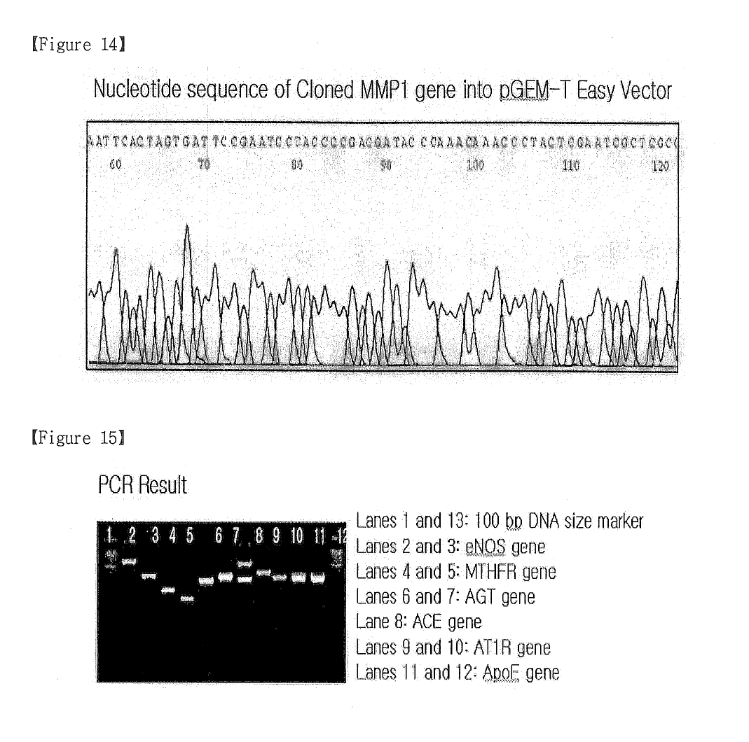

Sequencing PCR was performed to verify the insertion of the MMP1 gene product in the pGEM-T Easy vector, using ABI377 [FIG. 14]. Details are as follows.

1) In order to use the MMP1 gene product as template for sequencing, it is important to set an adequate concentration. In the present invention, 10 ng of MMP1 gene was used.

2) A forward or reverse primer (3.2 pmol) of MMP1 gene and terminator ready reaction mix (8 μL, Perkin Elmer, USA) were added to a PCR tube. After adding sterilized distilled water to a final volume of 20 μL, the mixture was mixed well.

3) Sequencing PCR was performed for the mixture using GeneAmp 2700 Thermal Cycler with 25 cycles of 96° C./10 sec, 50° C./5 sec and 60° C./6 min.

4) The reaction product was precipitated with ethanol and the free primer and fluorescence-labeled dideoxynucleotides (ddNTPs) in the terminator ready reaction mix were removed by centrifuge, followed by drying.

5) The resultant DNA was mixed with a mixture of formamide, 25 mM EDTA (pH 8.0) and blue dextran as well as loading buffer (10 μL). After denaturation in boiling water for 5 minutes, the sample was put on ice. The denaturation DNA sample was added to each well of a plate previously casted with 5.5% Long Ranger gel. Electrophoresis was performed for 2-4 hours and base sequence was analyzed using ABI377 automatic sequencer (Perkin Elmer, USA).

As a result, it was verified that the MMP1 gene was accurately amplified. It was confirmed that the gene product amplified in accordance with the present invention was inserted in the pGEM-T Easy vector and thus maintained stably. This indicates that the present invention is applicable to gene mutation tests and cancer detection.

### Example 13

### PCR-RFLP Using Skin Gene Card

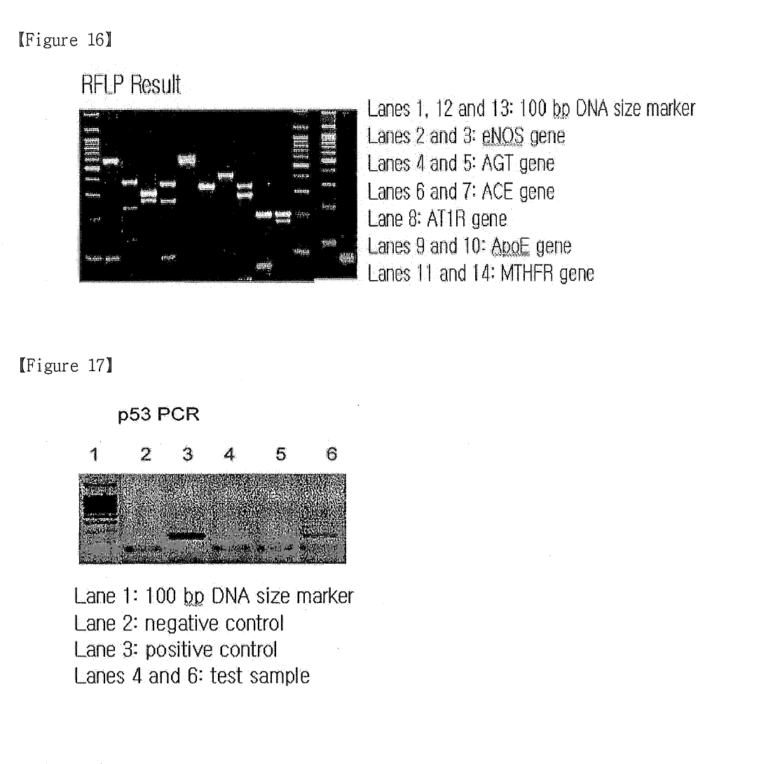

The skin genomic DNA sample acquired and stored using the skin gene card of the present invention was subjected to PCR followed by RFLP to verify whether genotyping test is possible. Genes involved in the onset of cardiovascular diseases were subjected to PCR, treated with specific restriction enzymes as follows, and then subjected to electrophoresis. The result revealed that multiple genotypes could be detected at once [FIGS. 15 and 16].

Details are as follows. The following 6 genes involved in adult diseases were acquired using the skin gene card of the present invention, and prepared into reaction solutions as in the following table in PCR tubes.

TABLE 1

| | eNOS1/2 | MTHFR1/2 | AGT1/2 | ACE1 | ACE2 | AT1R | APOE1/2 |
|---|---|---|---|---|---|---|---|
| D.W. | 20.8 | 20.8 | 19.3 | 19.1 | 17.6 | 20.3 | 17.8 |
| 10x buffer | 3 | 3 | 3 | 3 | 3 | 3 | 3 |
| 25 mM MgCl₂ | 2 | 2 | 2.5 | 2 | 2 | 3 | 2 |
| 2.5 mM dNTPs | 1 | 1 | 2 | 1.2 | 1.2 | 1.5 | 1 |
| F (10 pmole) | 1 | 1 | 1 | 1 | 1 | 0.5 | 1 |
| R (10 pmole) | 1 | 1 | 1 | 1 | 1 | 0.5 | 1 |
| DMSO | — | — | — | 1.5 | 3 | — | 3 |
| GenePol 5 U/L | 0.2 | 0.2 | 0.2 | 0.2 | 0.2 | 0.2 | 0.2 |
| Template DNA | 1 | 1 | 1 | 1 | 1 | 1 | 1 |
| Final volume | 30 | 30 | 30 | 30 | 30 | 30 | 30 |
| (ul) | | | | | | | |

The PCR tube holding the reaction solution was loaded on PE2700 Thermal Cycler (Perkin Elmer, USA) and gene amplification was performed as follows.

1. eNOS1/2, MTHFR1/2, AGT1/2, AT1R and ACE1 genes:

95° C./5 min, 35 cycles (95° C./30 sec, 58° C./30 sec, 72° C./40 sec), 72° C./10 min. ° C.

2. ACE2 and APOE1/2 genes:

95° C./5 min, 35 cycles (95° C./30 sec, 65° C./30 sec, 72° C./40 sec), 72° C./10 min.

The PCR product of each gene was subjected to electrophoresis on 1.2% agarose gel containing EtBr. The gene products and their sizes are summarized in Table 2.

TABLE 2

| Genes | Location | Size (bp) |
|---|---|---|
| ApoE | C112R | 330 |
| | R158C | 330 |
| AGT | M235T | 303 |
| | T174M | 354 |
| ACE | D/I | 319/597 |
| | D/D | 335/not amplified |
| ATIR | A1166C | 404 |
| eNOS | G10-T | 676 |
| | E298D | 371 |
| MTHFB | A222V | 198 |
| | A429E | 128 |

In order to perform RFLP of the resultant PCR products, the PCR products of five genes (eNOS, MTHFR, AGT, AT1R and APOE) excluding the ACE gene were purified using DNA Clean and Concentrator kit (Research Corporation, CA USA) as follows.

1. To the PCR product (˜25 uL), DNA binding solution (50 uL) is added.

2. The solution of 1. is transferred to Zymo spin column and centrifuged at 13,000 rpm for 30 seconds.

3. The solution collected at a collection tube is discarded using a pipette.

4. Washing buffer (200 uL) is added to the column and centrifugation is performed at 13,000 rpm for 30 seconds (twice).

5. Centrifugation is performed at 13,000 rpm for 40 seconds to completely remove the remaining washing buffer.

6. After removing the collection tube, a fresh 1.5 mL microcentrifuge tube is loaded to a fresh column. After adding sterilized triple distilled water (20 uL), centrifugation is performed at 13,000 rpm for 40 seconds for elution. Alternatively, sterilized triple distilled water heated to about 65° C. may be used.

For the resultant purified PCR products, restriction enzymes were prepared as in Table 3. Under a reaction condition adequate for each restriction enzyme, incubation was performed at 37° C. for 4-6 hours. Then, the risk factor for each gene was monitored through 2.5% agarose gel electrophoresis.

TABLE 3

| Genes | Location | Enzymes |
|---|---|---|
| ApoE | C112R | AflIII |
| | R158C | HaeII |
| AGT | M235T | LweI |
| | T174M | NcoI |
| ATIB | A1166C | DdeI |
| eNOS | G10-T | HincII |
| | E298D | Eco24I |
| MTHFB | A222V | HinfI |
| | A429E | MboII |

### Example 14

### Automated Base Sequencing of Sample Acquired from Skin Gene Card

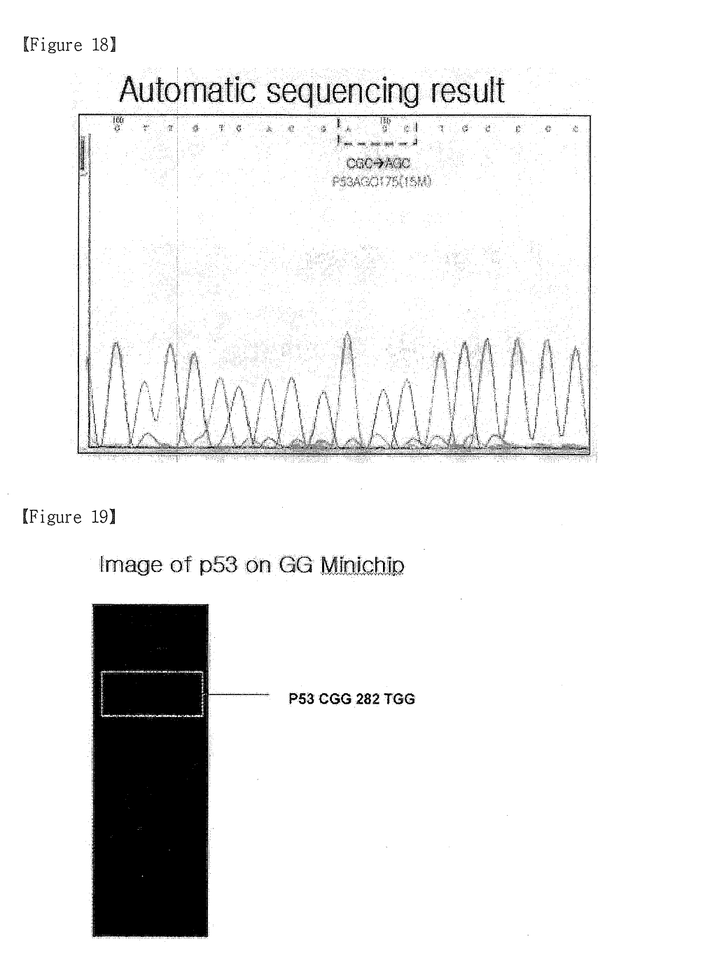

Squamous cell carcinoma skin sample was acquired and stored using the skin gene card of the present invention. It was verified whether genotyping is possible by automated base sequencing of the genomic DNA sample. p53 tumor suppressor gene, which plays an important role in carcinogenesis, was subjected to PCR and automated base sequencing was performed as follows. It was verified that detection of mutation of p53 can be carried out without any problem [FIGS. 17 and 18, Table 4].

Details are as follows.

In order to identify the mutation of p53 tumor suppressor gene using the skin gene card of the present invention, a reaction solution was prepared in a PCR tube as in Table 4.

TABLE 4

| Composition | ul |
|---|---|
| D.W. | 20.8 |
| 10x buffer | 3 |
| 25 mM MgCl₂ | 2 |
| 2.5 mM dNTPs | 1 |
| F (10 pmole) | 1 |
| R (10 pmole) | 1 |
| DMSO | — |
| GenePol 5 U/L | 0.2 |
| Template DNA | 1 |
| Final volume (uL) | 30 |

The PCR tube holding the reaction solution was mounted on PE2700 Thermal Cycler (Perkin Elmer, USA) and amplification was performed as follows: 94° C./5 min, 32 cycles (95° C./30 sec, 60° C./30 sec, 72° C./30 sec), 72° C./5 min.

The PCR product of the gene was subjected to electrophoresis on 1.2% agarose gel containing EtBr. Thus obtained PCR product was purified using DNA Clean and Concentrator kit (Research Corporation, CA USA) as follows.

1. To the PCR product (˜25 μL), DNA binding solution (50 μL) is added.

2. The solution of 1. is transferred to Zymo spin column and centrifuged at 13,000 rpm for 30 seconds.

3. The solution collected at a collection tube is discarded using a pipette.

4. Washing buffer (200 μL) is added to the column and centrifugation is performed at 13,000 rpm for 30 seconds (twice).

5. Centrifugation is performed at 13,000 rpm for 40 seconds to completely remove the remaining washing buffer.

6. After removing the collection tube, a fresh 1.5 mL microcentrifuge tube is loaded to a fresh column. After adding sterilized triple distilled water (20 μL), centrifugation is performed at 13,000 rpm for 40 seconds for elution. Alternatively, sterilized triple distilled water heated to about 65° C. may be used.

For the resultant purified PCR product of p53 gene, base sequencing was performed using ABI 3130 (Applied Biosystems) automatic sequencer.

### Example 15

### Genotyping of Sample Acquired Using Skin Gene Card by Way of Oligonucleotide Microarray

Squamous cell carcinoma skin sample was acquired and stored using the skin gene card of the present invention as in Example 13. It was verified whether genotyping is possible by way of oligonucleotide microarray of the RNA sample. p53 tumor suppressor gene, which plays an important role in carcinogenesis, was subjected to PCR and automated base sequencing was performed as follows. It was verified that detection of mutation of p53 can be carried out without any problem [FIG. 18].

Details are as follows. CanScan DNA chip (Goodgene) was used.

First, cDNA was synthesized using the RNA acquired from the skin gene card according to a known method. Then, p53 gene was amplified using PCR premix included in CanScan DNA chip. The PCR product was placed on the CanScan DNA chip and mini-sequencing was carried out. The result was analyzed using a fluorescence scanner [FIG. 19].

1. Amplification of p53 Gene:

Premix for PCR was prepared as follows.

| | | |
|---|---|---|
| Template cDNA | ~500 | ng |
| Taq DNA polymerase | 0.1 | uL |
| Primer set for each mutation site | 1 | uL |
| MgCl₂ | 1 | uL |
| 10x buffer | 3 | uL |
| 2.5 mM dNTPs | 1 | uL |
| G solution | 4 | uL |
| Distilled water | To 30 | uL |
| Final volume | 30 | uL |

PCR was performed using the prepared premix and a PCR machine (PE2700) under the following conditions.

| | | Temperature (° C.)/time (sec) | | | | | | Cycle |
|---|---|---|---|---|---|---|---|---|
| | Stage | 1M | 3M | 5M | 7M | 11M | 13M | (s) |
| 1 | Predenaturation | 95/300 | 95/300 | 95/300 | 95/300 | 95/300 | 95/300 | 1 |
| 2 | Denaturation | 95/30 | 94/30 | 95/30 | 94/40 | 94/40 | 95/30 | 40 |
| 3 | Annealing | 66/30 | 66/20 | 66/20 | 62/30 | 64/50 | 66/20 | |
| 4 | Elongation | 72/40 | 72/30 | 72/30 | 72/40 | 72/40 | 72/30 | |
| 5 | Final elongation | 72/420 | 72/420 | 72/420 | 72/420 | 72/420 | 72/420 | 1 |

| | | |
|---|---|---|
| 6 | Storage | 4/8 |

2. Fragmentation of PCR Product for Mini-Sequencing

The resultant PCR product was transferred to a fresh PCR tube and a reaction solution was prepared as follows.

| | | |
|---|---|---|
| 1 | Purified PCR product | 15 uL |
| 2 | U1 (0.1 U/ul) | 1 uL |
| 3 | U2 (0.1 U/ul) | 1 uL |
| 4 | 10x U buffer | 3 uL |
| 5 | Sterilized deionized water | 10 uL |
| | Total volume | 30 uL |

Thus prepared mixture was incubated at 37° C. for 1 hour. After boiling at 95° C. for 10 minutes, the mixture was stored in ice.

3. Mini-Sequencing

The fragmented PCR product (10 uL) was transferred to a fresh PCR tube. After adding distilled water (50 uL), followed by denaturation at 95° C. for 10 minutes, the tube was placed on ice. A reaction solution was prepared as follows in another PCR tube. The reaction solution was mixed well with the denatured, fragmented PCR product.

| | | | |
|---|---|---|---|
| 1 | 10x reaction buffer | 12 | uL |
| 2 | 5 uM ddATP | 1 | uL |
| 3 | 5 uM ddCTP | 1 | uL |
| 4 | 5 uM ddGTP | 1 | uL |
| 5 | 5 uM Cy5-ddUTP | 1 | uL |
| 6 | Sequenase (2 U/ul) | 1 | uL |
| 7 | Sterilized deionized water | 40 | uL |
| 8 | Total volume | 60 | uL |

The prepared mixture was slowly injected into the hole of the chip. Then, the chip was loaded on a hybridization chamber and incubated at 58° C. for 20 minutes. After washing with washing buffer I and II according to a known method, the signal was analyzed using a fluorescence scanner.

### Example 16

### Northern Blotting Analysis of Sample Acquired from Skin Gene Card

Northern blotting was performed for the RNA acquired from the skin gene card in Example 8 in order to identify expression of specific gene.

1) The RNA sample, 5× formaldehyde gel-running buffer (0.1 M MOPS, pH7.0: 40 mM sodium acetate: 5 mM EDTA, 2 ul), formaldehyde (3.5 ul) and formamide (10 ul) were added to a microfuge tube. H20 was added to a final volume of 20 ul.

2) The mixture was incubated at 65° C. for 15 minutes and put on ice for 5 minutes. After centrifuging for 5 seconds, the solution was mixed with formaldehyde gel-loading dye (2 ul).

3) Agarose gel was added to a gel running tank containing 1× formaldehyde gel-running buffer, and pre-run was made at 5 V for 5 minutes.

4) The agarose gel had been prepared by completely dissolving agarose (0.6 g) in DEPC-DW (31.1 mL), cooling to about 60° C., and then adding 5× formaldehyde gel-running buffer (10 mL) and formaldehyde solution (8.9 mL).

5) The sample was loaded on agarose gel and run was made at 3 V/cm.

6) When the sample moved about 8 cm, the gel was withdrawn and immersed in 0.1 M ammonium acetate solution containing 0.5 ug/mL ethidium bromide.

7) 30 minutes later, after taking pictures under UV, the gel was placed between NC filter that had been previously immersed in 6×SSC buffer and 3 MM paper (3 MM paper-gel-NC filter-3 MM paper-paper towel).

8) After transference for 18 hours, the NC filter was immersed in 6×SSC buffer and dried for 30 minutes at room temperature.

9) The NC filter was placed between 3 MM paper and baked for 2 hours in an 80° C. vacuum oven. Then, the NC filter was subjected to pre-hybridization for 2 hours at 42° C. (pre-hybridization buffer: 50% formamide, 5×SSPE, 5×Denhardt's solution, 0.1% SNS, 100 ug/mL denatured salmon sperm DNA).

10) After adding a probe for MMP1 gene labeled with a radioactive isotope to the hybridization solution, reaction was carried out for 16 hours at 42° C.

11) The NC filter was washed 2 times with 2×SSC and 0.1% SDS buffer for 5 minutes each, and dried at room temperature.

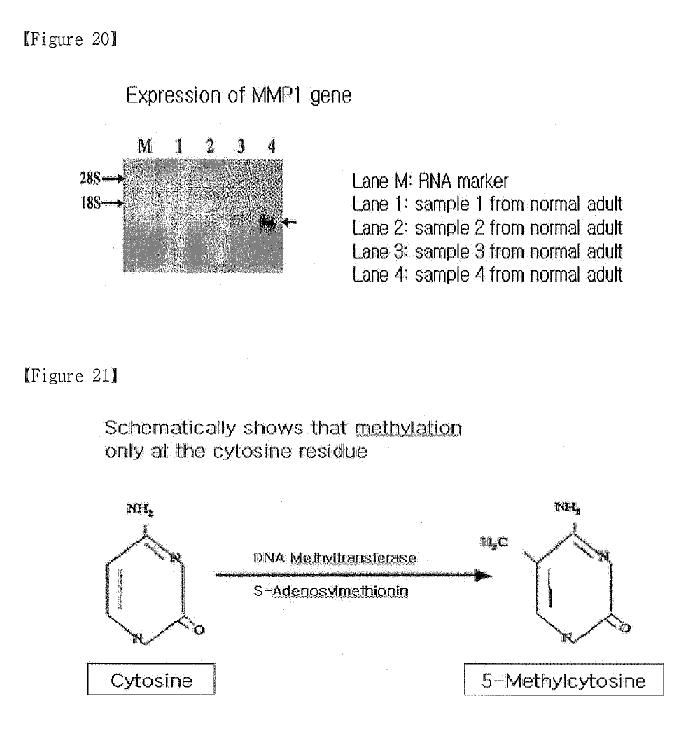

12) The dried NC filter was exposed to X-ray film to detect the gene expression [FIG. 20].

### Example 17

### Analysis of Promoter Methylation in DNA Sample Acquired from Skin Gene Card by MSP

DNA methylation in higher eukaryotes occurs only at the 5′-site of the cytosine residue of CpG dinucleotide [FIG. 21]. Since this change occurs mainly at the CpG-rich portion of the promoter called “CpG island”, it is important in the regulation of gene expression. Hypermethylation at the CpG island inactivates the expression of specific genes, which is known to occur frequently in human tumor suppressor genes. The inactivation of tumor suppressor genes ultimately leads to carcinogenesis.

In order to establish a MSP method of the DNA sample acquired from the skin gene card by analyzing promoter methylation of genes, the following experiment was performed.

1) First, the DNA acquired from the skin gene card was treated with CpGenome(tm) DNA modification kit (Cat. No. S7820, Intergen Co., NY), containing sodium bisulfite as main component, to convert unmethylated cytosine into uracil.

2) MSP is a technique selecting two primer sets on an assumption of two template base sequences (i.e. methylated and unmethylated) based on the fact that the cytosine residue in the genome is converted into uracil or not upon treatment with sodium bisulfite depending on whether it is already methylated, and evaluating methylation from the PCR amplification profile. In this example, primer sets capable of amplifying unmethylated sequence were used.

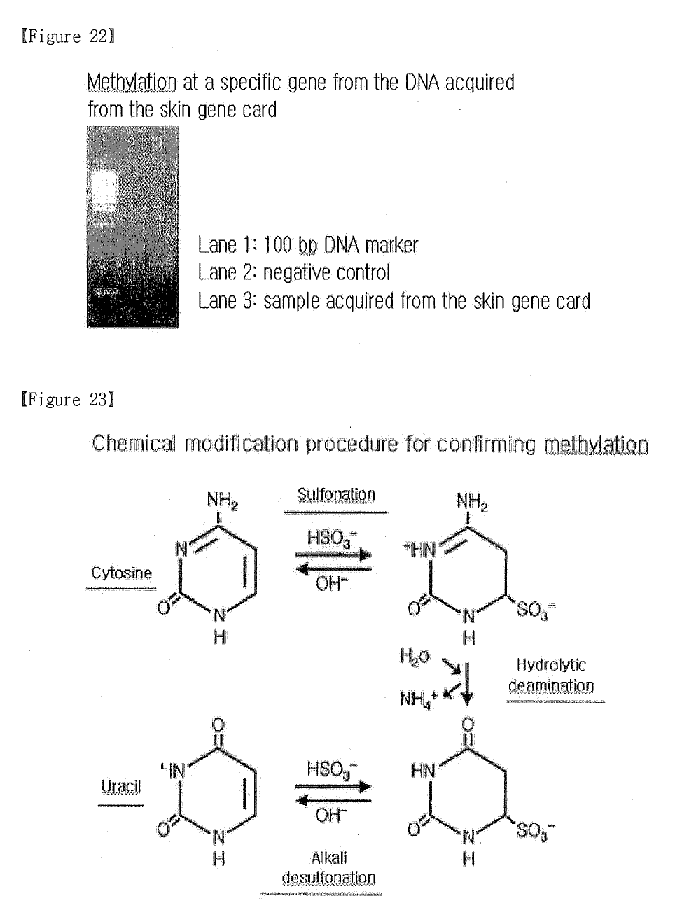

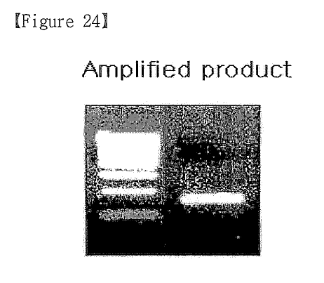

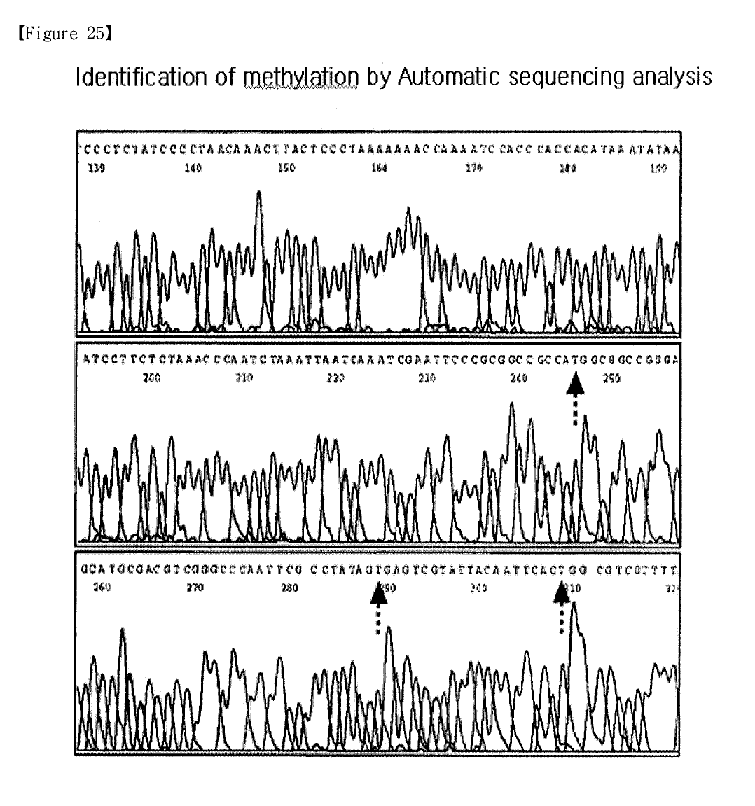

3) PCR was performed using denatured DNA as template and gene amplification was identified [FIG. 22].

The result suggests that methylation of specific genes can be verified through MSP for the DNA sample acquired using the skin gene card.

### Example 18

### Analysis of Promoter Methylation in DNA Sample Acquired from Skin Gene Card by Bisulfite Genomic Sequencing

Bisulfite genomic sequencing was performed for using the DNA sample acquired from the skin gene card as another method for analysis of promoter methylation of specific genes. When DNA is chemically treated with sodium bisulfite, the cytosine residue of the DNA base sequence is converted into uracil. When the product is subjected to PCR, unmethylated cytosine is converted to thymine. Hence, the site of methylation can be detected. Details are as follows. First, chemical modification was carried out as follows using DNA methylation kit (Zymo).

1. M-Dilution buffer (10 ul) is added to DNA solution (90 ul) and the mixture is incubated at 37° C. for 15 minutes.

2. After adding 200 ul of CT conversion reagent solution (750 ul D.W. and 210 ul M-dilution buffer are completely mixed by vortexing), the mixture is gently shaken for incubation at 50° C. for 16 hours (The remaining CT conversion reagent solution may be stored at −20° C. for reuse within a week).

3. After incubation on ice for 10 minutes, M-binding buffer (800 ul) is added.

4. The mixture (600 ul) is loaded into Zymo-Spin I column and centrifuged at 25° C. and 11,000 rpm (Eppendorf centrifuge) for 1 minute.

5. After discarding waste away from the collection, the remaining sample is loaded and centrifuged at 25° C. and 11,000 rpm (Eppendorf centrifuge) for 1 minute.

6. After discarding waste away from the collection followed by loading of M-wash buffer (200 ul), centrifugation is performed at 25° C. and 11,000 rpm (Eppendorf centrifuge) for 1 minute.

7. After loading M-Desulphonation buffer (200 ul), incubation is performed at room temperature for 15 minutes.

8. Centrifugation is performed at 25° C. and 11,000 rpm (Eppendorf centrifuge) for 1 minute.

9. After loading M-wash buffer (200 ul), centrifugation is performed at 25° C. and 11,000 rpm (Eppendorf centrifuge) for 1 minute.

10. After transferring the column to a collection tube followed by loading of M-wash buffer (200 ul), centrifugation is performed at 25° C. and 13,000 rpm (Eppendorf centrifuge) for 1 minute.

11. After loading prewarmed M-elution buffer (90 ul, 70° C.) to a column, the column is transferred to a 1.5 mL tube. 1 minute later, centrifugation is performed at 25° C. and 11,000 rpm (Eppendorf centrifuge) for 2 minutes to elute the modified DNA.

The modified DNA was prepared into a reaction solution as follows and amplified using Thermal Cycler.

TABLE 5
Composition of amplification reaction
solution and amplification condition

| PCR reaction mix (ul) | | PCR cycle | | |
|---|---|---|---|---|
| 10x buffer | 2.5 | 95° C. | 5 min | |
| Ea 2.5 mM dNTPs | 2 | | | |
| 25 mM MgCl₂ | 1.5 | | | |
| 20 uM MYOD-F primer | 0.5 | 94° C. | 30 sec | 40 cycles |
| 20 uM MYOD-R primer | 0.5 | 61° C. | 40 sec | |
| 5 U/ul AmpliTaq Gold | 0.1 | 72° C. | 40 sec | |
| Template DNA | 3 | | | |
| D.W. | 15 | | | |
| Total | 25 | 72° C. | 7 min | |

The amplification product was subjected to 2% agarose gel electrophoresis.

DNA was acquired from the amplification product by cutting out of the agarose gel and was purified as follows using DNA Clean and Concentrator kit (Zymo Research Corporation, CA USA).

1. The PCR product (˜25 μL) is mixed with DNA binding solution (50 μL).

2. The solution of 1. is transferred to Zymo spin column and centrifuged at 13,000 rpm for 30 seconds.

3. The solution collected at a collection tube is discarded using a pipette.

4. Washing buffer (200 μL) is added to the column and centrifugation is performed at 13,000 rpm for 30 seconds (twice).

5. Centrifugation is performed at 13,000 rpm for 40 seconds to completely remove the remaining washing buffer.

6. After removing the collection tube, a fresh 1.5 mL microcentrifuge tube is loaded to a fresh column. After adding sterilized triple distilled water (20 μL), centrifugation is performed at 13,000 rpm for 40 seconds for elution. Alternatively, sterilized triple distilled water heated to about 65° C. may be used.

The purified DNA was sequenced using a base sequencer to identify methylation (cytosine→thymine) at specific sites.

### Example 19

### Personal Identification Using Sample Acquired from Skin Gene Card

Human chromosomal DNA has tandem repeat sequences. Among the repeat sequences, those of 14-70 bp are called variable number of tandem repeats (VNTRs) and those of 2-7 bp are called short tandem repeats (STRs). The VNTR or STR is a polymorphism occurring when a pattern of two or more nucleotides are repeated and the repeated sequences are directly adjacent to each other. Since they are different in length between individuals, they can be used for personal or parental identification. In this example, the VNTR gene loci D1S80 and D17S30, and the STR gene loci D3S1358, D5S818, D7S820, D8S1179, D135317, D18551, D21S11, FGA and vWA were examined.

19-1. Multiplex PCR of VNTR and STR Loci

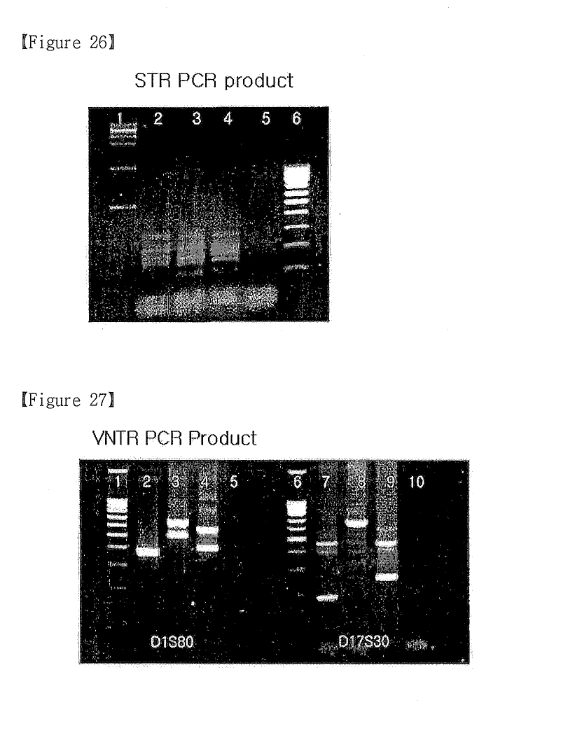

Genomic DNA was extracted from the skin sample acquired using the skin gene card. Then, specific genes were amplified by Multiplex-PCR using VNTRs-PCR and AmpF1 STR Profiler Plus PCR amplification kit (Applied Biosystems). The PCR result revealed that DNA could be adequately acquired from the skin sample and analyzed [FIGS. 26 and 27].

19-2. Personal Identification

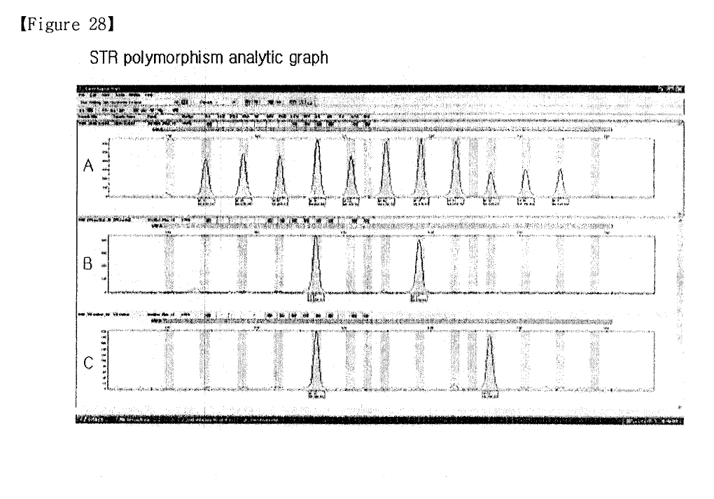

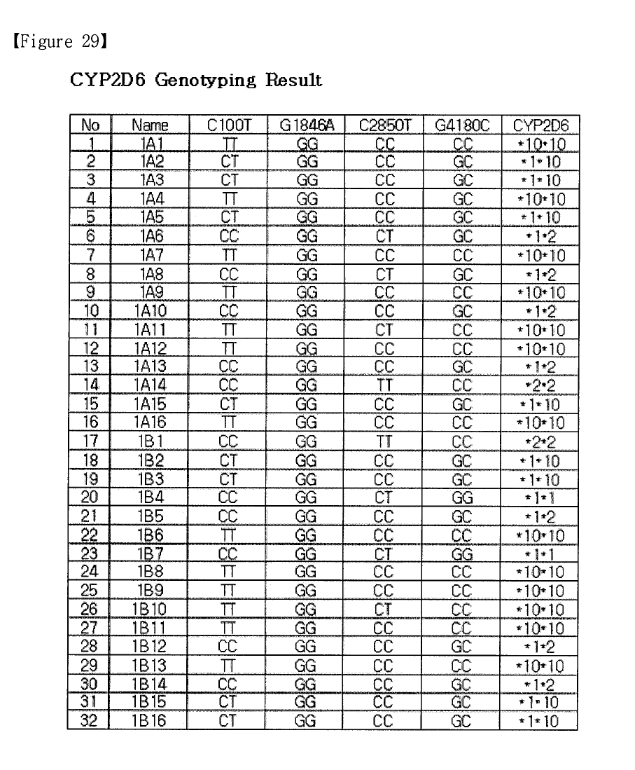

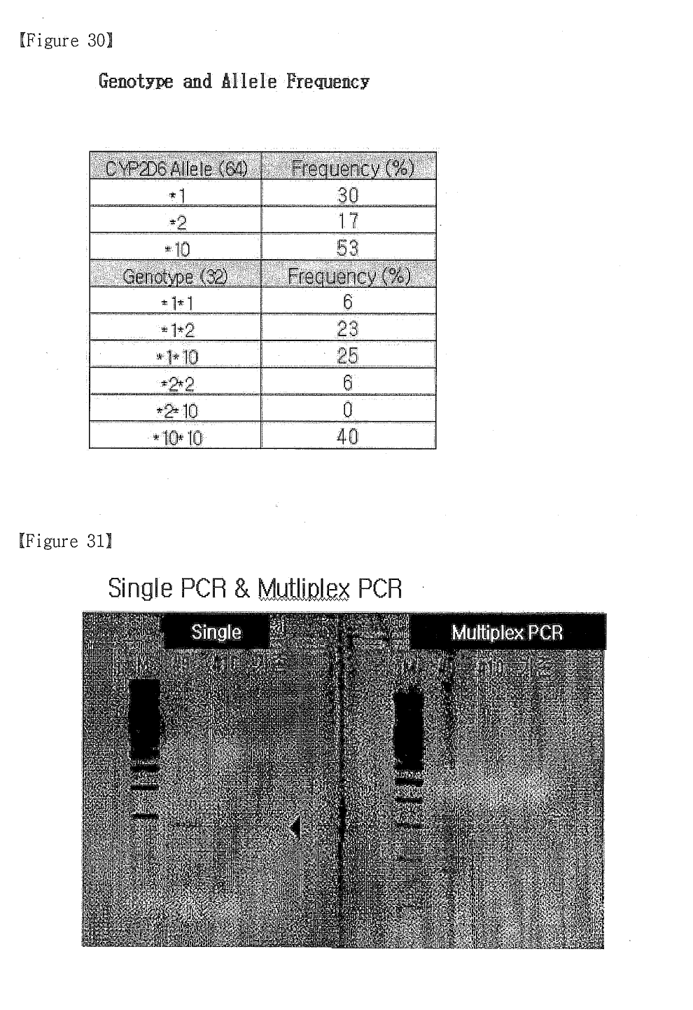

From the acquired genomic DNA, STRs were obtained using AmpF1 STR Profiler Plus PCR amplification kit, sequenced using ABI 3130xl Genetic analyzer (Applied Biosystems) and analyzed using GeneMapper ID program (Human Identification Detecton, Applied Biosystems) [FIG. 28].

### Example 20

### Pharmacogenomic Test of Sample Acquired from Skin Gene Card Using Base Sequencer

The understanding of single-nucleotide polymorphism (SNP) of individuals allows the understanding of response and adverse reactions to specific drugs of the individuals. The so-called “pharmacogenomic test” is of help in drug development, selection of personalized drugs, and minimization of adverse reactions to drugs. In this example, it was verified whether SNP analysis of representative genes involved in drug metabolism is possible for the sample acquired using the skin gene card. Details are as follows. The result indicates that SNP analysis of genes involved in drug metabolism is possible with the sample acquired using the skin gene card, and that it can be of help in predicting response and adverse reactions to specific drugs of an individual, selecting personalized drugs, and minimizing adverse reactions to drugs.

20-1. CYP2D6 Genotyping

Genomic DNA was extracted from skin sample acquired using the skin gene card. Genotyping analysis was performed for CYP2D6 gene, a representative gene involved in drug metabolism. Genotypes and allele frequencies were obtained from the analysis. The result revealed that DNA can be adequately acquired from the skin sample and analyzed.

### Example 21

### Nutrigenomic Test of Sample Acquired from Skin Gene Card

21-1. Basic Principle

This test is based on the understanding of genetic polymorphism of an individual, such as mutation of genes involved in oxidative stress, liver detoxification, cardiovascular health, hormone metabolism and immuno-/osteo-health, through SNP assays based on genetic techniques (multiplex-PCR/SNaPshot Multiplex method).

21-2. Single- or Multiplex-PCR

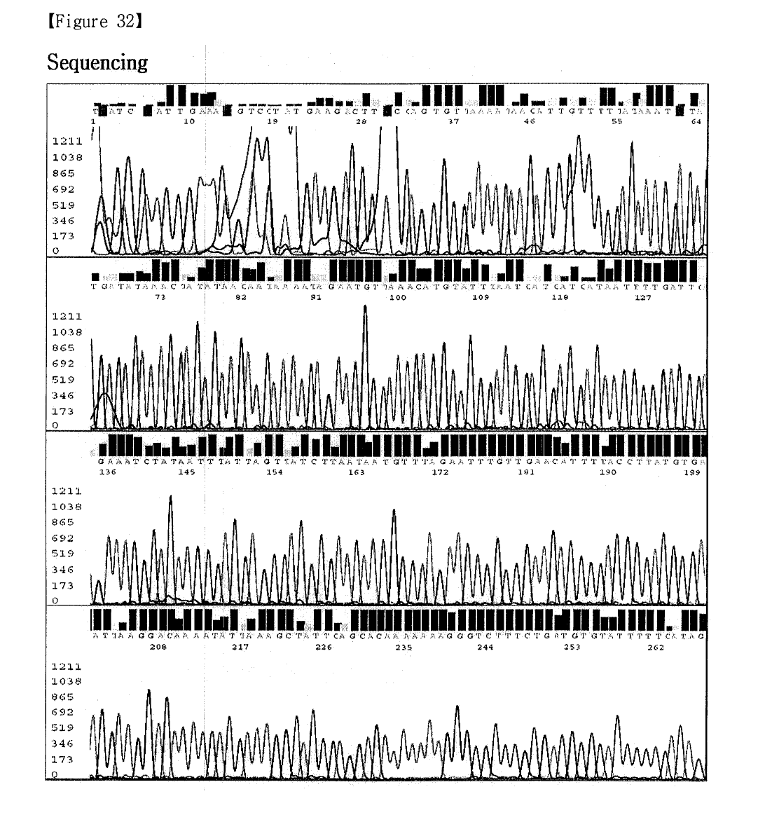

Genomic DNA was extracted from skin sample acquired using the skin gene card. Specific genes were amplified by PCR using specific single- and multiplex-PCR primers [FIG. 31]. The PCR result revealed that DNA can be adequately acquired from the skin sample and analyzed.

21-3. Sequencing and SNaPshot Multiplex Assay

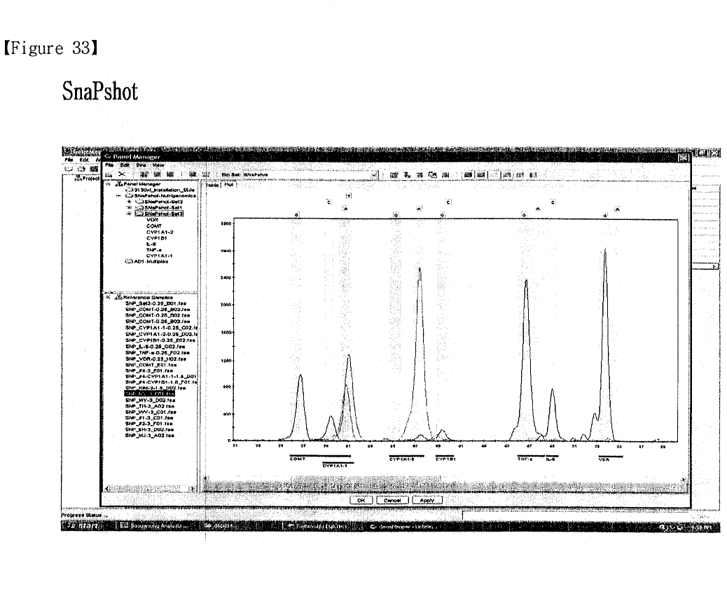

Using the acquired genomic DNA, SNP was determined using Sequencing and SNaPshot Multiplex kit (Applied Biosystems) and genotyping was carried out using ABI 3130xl Genetic analyzer (GeneMapper program) [FIGS. 32 and 33].

21-4. Analysis using Anti-Aging and Well being Chip

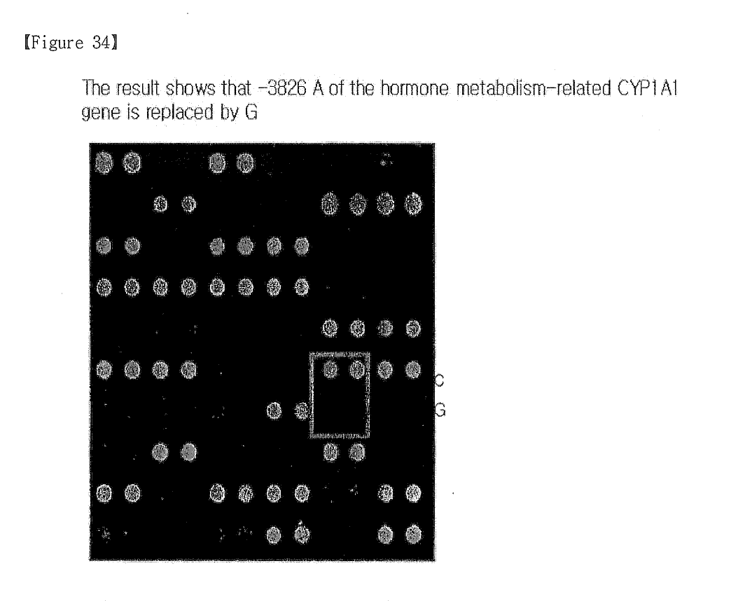

Using genomic DNA acquired as in Example 3 using the skin gene card, 18 nutrigenomic genes (genes involved in obesity, antioxidative stress, detoxification, cardiovascular disease, hormone metabolism, allergy and bone metabolism) were amplified by a known multiplex method. Analysis with AW (Anti-aging and Well being) chip (Goodgene) revealed that the nutrigenomics test could be carried out without any problem [FIG. 34]. Details are as follows.

1. Fragmentation of PCR Product for Mini-Sequencing

20 PCR products of 18 genes were prepared into reaction solutions in fresh PCR tubes.

| | | |
|---|---|---|
| 1 | Purified PCR product | 15 μL |
| 2 | U1 (0.1 U/ul) | 1 μL |
| 3 | U2 (0.1 U/ul) | 1 μL |
| 4 | 10x U buffer | 3 μL |
| 5 | Sterilized deionized water | 10 μL |
| | Total volume | 30 μL |

Thus prepared mixture was incubated at 37° C. for 1 hour. After boiling at 95° C. for 10 minutes, the mixture was stored in ice.

2. mini-sequencing

The fragmented PCR product (10 μL) was transferred to a fresh PCR tube. After adding distilled water (50 μL), followed by denaturation at 95° C. for 10 minutes, the tube was placed on ice. A reaction solution was prepared as follows in another PCR tube. The reaction solution was mixed well with the denatured, fragmented PCR product.

| | | | |
|---|---|---|---|
| 1 | 10x reaction buffer | 12 | μL |
| 2 | 5 uM ddATP | 1 | μL |
| 3 | 5 uM Cy3-ddCTP | 1 | μL |
| 4 | 5 uM ddGTP | 1 | μL |
| 5 | 5 uM Cy5-ddUTP | 1 | μL |
| 6 | Sequenase (2 U/ul) | 1 | μL |
| 7 | Sterilized deionized water | 40 | μL |
| 8 | Total volume | 60 | μL |

The prepared mixture was slowly injected into the hole of the chip. Then, the chip was loaded on a hybridization chamber and incubated at 58° C. for 20 minutes. After washing with washing buffer I and II according to a known method, the signal was analyzed using a fluorescence scanner.

### Example 22

### Diagnosis of Genetic Disease Using Sample Acquired from Skin Gene Card

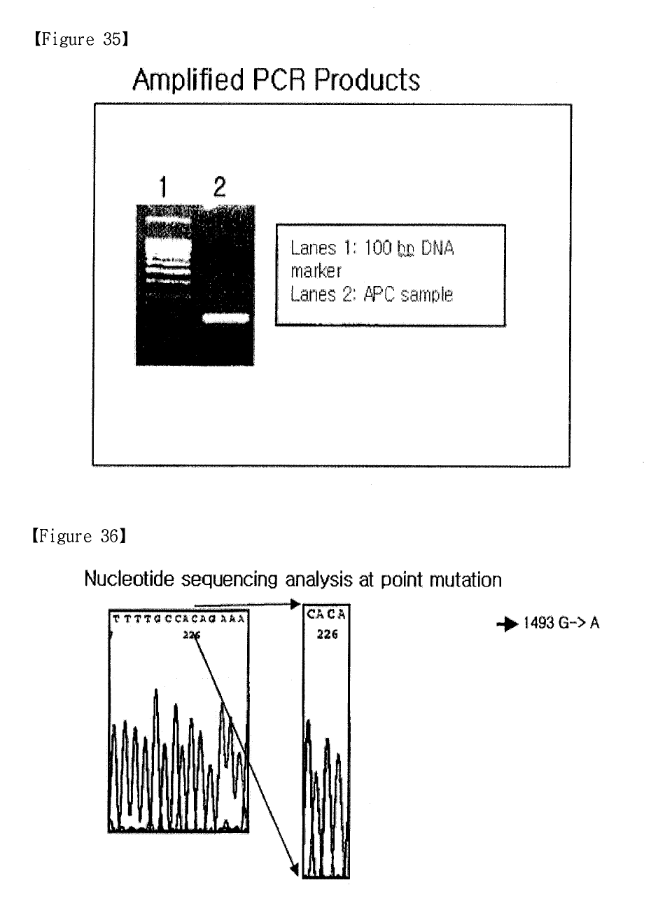

Genomic DNA was acquired from skin sample acquired using the skin gene card. Gene amplification was carried out through 40 cycles of PCR using a primer specific to APC gene. The amplification product was subjected to electrophoresis on agarose gel [FIG. 35]. DNA was acquired from the amplification product by cutting out of the agarose gel and was purified. The purified product was subjected to base sequencing using 3130 Sequence Analyze system to identify point mutation on the base sequence [FIG. 36].

### Example 23

### Diagnosis by Test of Skin Cancer-Related Genes Using Sample Acquired from Skin Gene Card

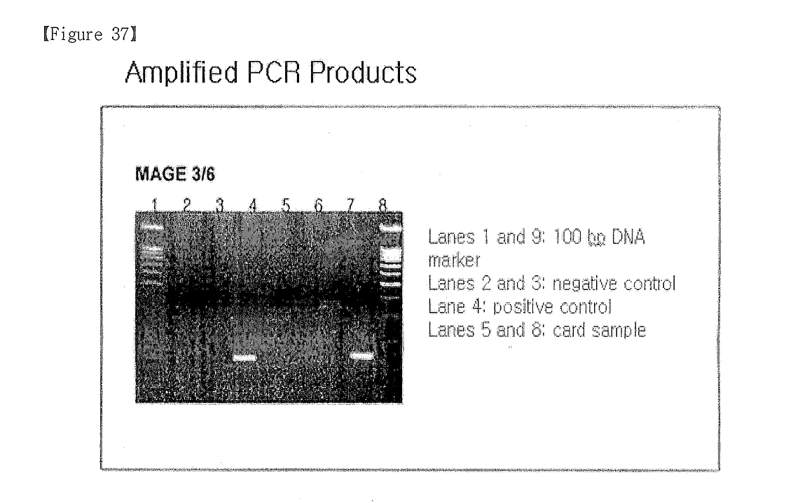

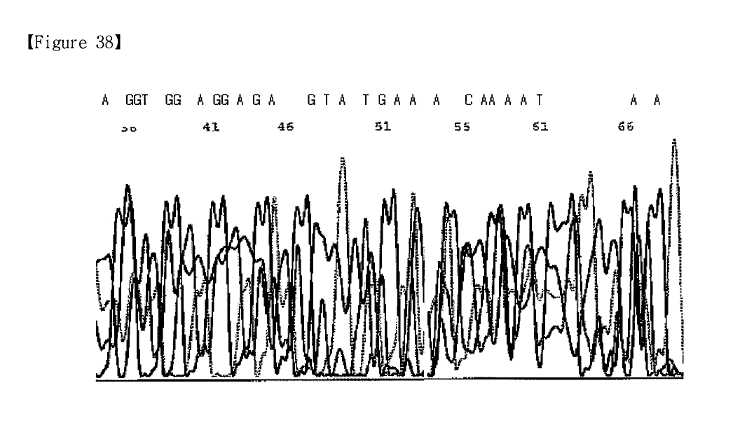

Tissue was acquired from the tumor of a melanoma patient using the skin gene card of the present invention. After extracting RNA therefrom using RNA extraction kit (iNtRON), cDNA was synthesized and expression of MAGE gene and the house-keeping gene β-actin was identified through PCR. Primers specific to the genes were used and the expression of melanoma antigen (MAGE) was identified through 40 cycles of PCR [FIG. 37].

### Example 24

### Diagnosis by Test of Skin Infection-Related Genes Using Sample Acquired from Skin Gene Card

Staphylococcus aureus, particularly methicillin-resistant staphylococcus (MSR)/pustular folliculitis, sycosis, atopy, tetracycline resistance: PCR/Sequencing/Chip

DNA was acquired from the sample acquired using the skin gene card. It was verified whether infectious disease can be detected using Staphylococcus aureus PCR kit (Goodgene). The result revealed that infectious disease can be detected without any problem. Details are as follows.

1. DNA Separation from Skin Gene Card

1) The sample (1.5 mL) is transferred to a 1.5 mL tube and loaded in a microcentrifuge. Centrifuge is performed at 12,000 rpm for 2 minutes so that the cells are sedimented.

2) After removing the supernatant, 1×PBS (500 μL) is added.

3) The cells are mixed well with the solution under vortex.

4 Centrifuge is performed at 12,000 rpm for 2 minutes and the supernatant is removed.

5) Buffer TL (200 μL) is added.

6) After adding protease K (20 μL), the mixture is mixed well under vortex.

7) The mixture is incubated at 56° C. for 30 minutes.

8) After completion of reaction, the tube is spun down at 8,000 rpm or above for about 10 seconds so that the solution adhering to the lid is dropped.

9) Buffer TB (400 μL) is added and mixed well. The tube is spun down at 8,000 rpm or above for about 10 seconds so that the solution adhering to the lid is dropped.

10) A spin column is equipped at a collection tube, and the above reaction solution is added to the spin column.

11) Centrifuge is performed at 8,000 rpm for 1 minute.

12) The filtrate passing through the column is discarded and another collection tube is mounted.

13) After adding buffer BW (700 μL), centrifuge is performed at 8,000 rpm for 1 minute.

14) The filtrate passing through the column is discarded and another collection tube is mounted.

15) After adding buffer NW (500 μL), centrifuge is performed at 12,000 rpm for 3 minutes.

16) The filtrate passing through the column is discarded and a fresh 1.5 mL tube is mounted.

17) After adding buffer AE (200 μL) or purified water at the middle portion of the column, the tube is left for 2 minutes at room temperature.

18) Centrifuge is performed at 8,000 rpm for 1 minute.

19) The extracted genomic DNA is subjected to PCR immediately or stored at −20° C. for later use.

20) The extracted genomic DNA is subjected to electrophoresis on 0.8% agarose gel at 100 V and examined under UV.

2. Identification of Infection by Staphylococcus aureus Through PCR

1) 2× master mix (12.5 μL) and primer mix (2.5 μL) were added to a PCR tube. Template DNA (10 μL) was added to a final volume of 25 μL and mixed well.

| | | | |
|---|---|---|---|
| Predenaturation | 95° C. | 10 min | 1 cycle |
| Denaturation | 95° C. | 40 sec | 40 cycles |
| Annealing | 60° C. | 40 sec | |
| Elongation | 72° C. | 40 sec | |
| Final elongation | 72° C. | 5 min | 1 cycle |

2) PCR was performed using the prepared premix and a PCR machine.

3) Upon completion of the reaction, the PCR product (5 μL) was subjected to electrophoresis on 2% agarose gel. 228 bp product was identified. Sequencing analysis was performed for the product (FIG. 38).

### Example 25

### Diagnosis by Test of Sexually Transmitted Disease-Related Genes Using Sample Acquired from Skin Gene Card

DNA was acquired from samples (skin, oral mucosa, vagina and anus) acquired using the skin gene card. Sexually transmitted disease was identified using 12 STD Multiplex PCR kit (Goodgene). It was verified that STD could be detected without any problem. Details are as follows.

1. DNA Separation from Skin Gene Card

1) The sample (1.5 mL) is transferred to a 1.5 mL tube and loaded in a microcentrifuge. Centrifuge is performed at 12,000 rpm for 2 minutes so that the cells are sedimented.

2) After removing the supernatant, 1×PBS (500 μL) is added.

3) The cells are mixed well with the solution under vortex.

4 Centrifuge is performed at 12,000 rpm for 2 minutes and the supernatant is removed.

5) Buffer TL (200 μL) is added.

6) After adding protease K (20 μL), the mixture is mixed well under vortex.

7) The mixture is incubated at 56° C. for 30 minutes.

8) After completion of reaction, the tube is spun down at 8,000 rpm or above for about 10 seconds so that the solution adhering to the lid is dropped.

9) Buffer TB (400 μL) is added and mixed well. The tube is spun down at 8,000 rpm or above for about 10 seconds so that the solution adhering to the lid is dropped.

10) A spin column is equipped at a collection tube, and the above reaction solution is added to the spin column.

11) Centrifuge is performed at 8,000 rpm for 1 minute.

12) The filtrate passing through the column is discarded and another collection tube is mounted.

13) After adding buffer BW (700 μL), centrifuge is performed at 8,000 rpm for 1 minute.

14) The filtrate passing through the column is discarded and another collection tube is mounted.

15) After adding buffer NW (500 μL), centrifuge is performed at 12,000 rpm for 3 minutes.

16) The filtrate passing through the column is discarded and a fresh 1.5 mL tube is mounted.

17) After adding buffer AE (200 μL) or purified water at the middle portion of the column, the tube is left for 2 minutes at room temperature.

18) Centrifuge is performed at 8,000 rpm for 1 minute.

19) The extracted genomic DNA is subjected to PCR immediately or stored at −20° C. for later use.

20) The extracted genomic DNA is subjected to electrophoresis on 0.8% agarose gel at 100 V and examined under UV.

2. PCR using STD Multiplex PCR Kit

Set A (UU, MH, CTR, TV, MG and NG mix)

Set B (HD, GV, TP, HSV, CA and HPV mix)

1) 2× master mix (12.5 μL) was added to a PCR tube.

2) After adding STD primer A (or B) set (4.5 μL) and genomic DNA (3 μL), distilled water was added to a final volume of 25 μL. After mixing well, PCR was performed using a PCR machine under the following conditions.

3) Denaturation (94° C., 15 min); 40 cycles of 94° C./30 sec, 58° C./1.5 min, 72° C./1.5 min; reaction (72° C./10 min).

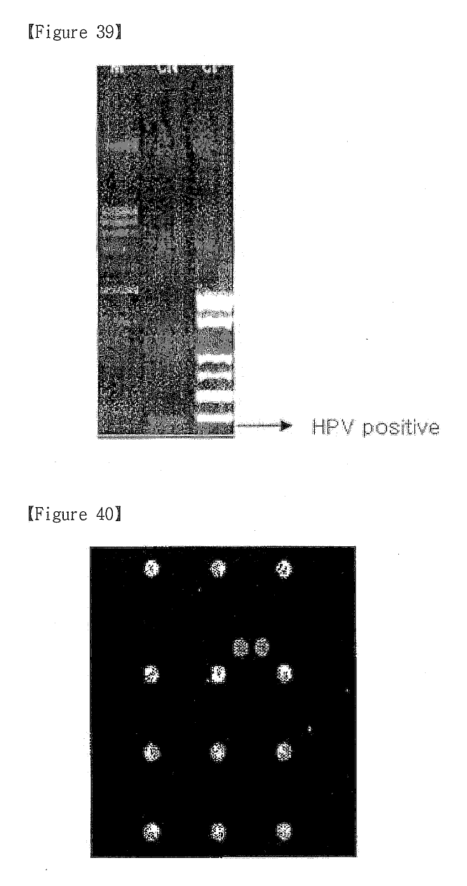

4) The PCR product (7-8 μL) was subjected to electrophoresis on 2% agarose gel and examined under UV [FIG. 39].

| | Predicted | | Predicted |
|---|---|---|---|
| Set A | size (bp) | Set B | size (bp) |
| U. urealyticum (UU) | 869 | H. ducreyi (HD) | 496 |
| M. hominis (MH) | 502 | G. vaginitis (GV) | 419 |
| C. trachomatis | 367 | T. pallidum (TP) | 313 |
| T. vaginalis (TV) | 319 | H. simplex virus (HSV) | 268 |
| M. genitalium (MG) | 253 | C. albicans (CA) | 234 |
| N. gonorrhoeae (NG) | 214 | Human papillomavirus | 185 |
| | | (HPV) | |

### Example 26

### Diagnosis by Test of Viral Infection-Related Genes Using Sample Acquired from Skin Gene Card

DNA was acquired from samples (skin, oral mucosa, vagina and anus) acquired using the skin gene card. Viral infection was identified using STD Multiplex PCR kit (Goodgene). It was verified that viral infection could be detected without any problem [FIG. 39]. Infection by HPV was identified using GG HPV genotyping chip (Goodgene) [FIG. 40]. Details are as follows.

1. DNA Separation from Skin Gene Card

1) The sample (1.5 mL) is transferred to a 1.5 mL tube and loaded in a microcentrifuge. Centrifuge is performed at 12,000 rpm for 2 minutes so that the cells are sedimented.

2) After removing the supernatant, 1×PBS (500 μL) is added.

3) The cells are mixed well with the solution under vortex.

4 Centrifuge is performed at 12,000 rpm for 2 minutes and the supernatant is removed.

5) Buffer TL (200 μL) is added.

6) After adding protease K (20 μL), the mixture is mixed well under vortex.

7) The mixture is incubated at 56° C. for 30 minutes.

8) After completion of reaction, the tube is spun down at 8,000 rpm or above for about 10 seconds so that the solution adhering to the lid is dropped.

9) Buffer TB (400 μL) is added and mixed well. The tube is spun down at 8,000 rpm or above for about 10 seconds so that the solution adhering to the lid is dropped.

10) A spin column is equipped at a collection tube, and the above reaction solution is added to the spin column.

11) Centrifuge is performed at 8,000 rpm for 1 minute.

12) The filtrate passing through the column is discarded and another collection tube is mounted.

13) After adding buffer BW (700 μL), centrifuge is performed at 8,000 rpm for 1 minute.

14) The filtrate passing through the column is discarded and another collection tube is mounted.

15) After adding buffer NW (500 μL), centrifuge is performed at 12,000 rpm for 3 minutes.

16) The filtrate passing through the column is discarded and a fresh 1.5 mL tube is mounted.

17) After adding buffer AE (200 μL) or purified water at the middle portion of the column, the tube is left for 2 minutes at room temperature.

18) Centrifuge is performed at 8,000 rpm for 1 minute.

19) The extracted genomic DNA is subjected to PCR immediately or stored at −20° C. for later use.

20) The extracted genomic DNA is subjected to electrophoresis on 0.8% agarose gel at 100 V and examined under UV.

2. PCR for HPV Chip

1) A predetermined amount of purified water was added to each primer to completely dissolve the primer. The completely dissolved primer may be stored at −20° C. The addition amount of purified water is as follows.

TABLE 6
Amount of purified water added to HPV PCR primer

| | | Addition amount |
|---|---|---|
| No. | Primer | of purified water |
| 1 | L1 | 210 uL |
| 2 | L2 | 220 uL |
| 3 | H1 | 230 uL |
| 4 | H2 | 250 uL |

2) L2 and H2 primers are stored after covering with silver foil because they are susceptible to light since the end group is labeled with cyanine 5.

The composition of the reaction solution of each tube is as follows.

TABLE 7
Composition of reaction solution for HPV gene amplification

| Reagents | Volume (per each reaction) |
|---|---|
| Premix | 15 uL |
| Purified water | 8 uL |
| L1 primer | 1 uL |
| L2 primer | 1 uL |
| Template genomic DNA | 5 uL |

* PCR compositions for L1/L2 and H1/H2 are similar.

1) Two premixes (for L and H genes) per each sample are prepared on ice.

2) Two master mix tubes for L and H genes are prepared.

3) Purified water is added to each 1.5 mL master mix tube.

4) The corresponding primer sets (L1 and L2 sets, H1 and H2 sets) are added to each master mix tube, and mixed well.

5) The prepared L and H master mixtures (10 uL) are added to each premix tube.

6) Template genomic DNA (5 uL) is added to the premix tube and mixed well.

7) After spinning down for a while with a centrifuge, the mixture is subjected to PCR under the following conditions.

TABLE 8
PCR condition for HPV gene amplification

| Process | Temperature | Time | Cycles |
|---|---|---|---|
| Predenaturation | 94° C. | 5 min | 1 |
| Denaturation | 94° C. | 30 sec | 40 |
| Annealing | 50° C. | 30 sec | |
| Elongation | 72° C. | 30 sec | |
| Final elongation | 72° C. | 5 min | 1 |

(Optional) Identification of Amplified DNA

The amplified DNA may be identified by electrophoresis on 2% agarose gel.

3. HPV DNA Chip Reaction

1) Fresh 1.5 mL or 200 μL tubes are prepared as many as the number of reaction samples.

2) Purified water (50 μL) is added to each tube.

3) Of the HPV PCR product, L1 (10 μL) or H (5 μL) is added and mixed well.

4) The tube is kept in a heat block of 95° C. for 3 minutes.

5) The tube is kept on ice for 5 minutes.

6) The reaction tube is spun down for 30 seconds by centrifuge.

7) HYB I buffer (65 μL) is added to the tube and mixed well with a pipette.

8) The prepared reaction solution is slowly injected into the hole of the cover slip on the chip surface.
It is checked if there is any bubble between the chip and the reaction chamber. If there are bubbles, they are removed by squeezing with a gloved hand.

9) Chip hybridization is performed at 48° C. for 30 minutes.

(Post-Hybridization Washing)

1) Upon completion of the hybridization, the cover slip is removed from the chip using forceps.

2) After pouring washing buffer 1 in a jar, the chip is washed at room temperature for 2 minutes using an orbital shaker.
Alternatively, the washing may be performed by spraying the washing buffer from a squeeze bottle onto the chip surface for 2 minutes.
If the number of the reaction chip is 1, the chip may be put in a 50 mL conical tube containing the washing buffer (40 mL) and the tube may be shaken for 2 minutes.

3) After discarding the washing buffer and adding washing buffer 2, washing is performed for 2 minutes.

4) A spin dryer or an air compressor may be used to remove the buffer remaining on the chip after the washing (Alternatively, the buffer may be removed using KimWipes. However, the chip should not be touched with a finger.

4. Result Interpretation

1) If the SBRs of all the H spots are 2.5 or above and the SBRs of all the L1 spots are 2.5 or above, the result is positive.

2) If the SBR of HBB is 2.5 or above and the SBR of only one of the two L1 spots is 2.5 or above, the test is performed again.

3) If the SBRs of all the HBB spots do not exceed 2.5, the test is performed again from the sampling.

4) If the result for only one spot is positive or negative, the test is performed again.

### Example 27

### Diagnosis by Test of Tuberculosis Infection-Related Genes Using Sample Acquired from Skin Gene Card

Recently, the prevalence of tuberculosis is on the increase again. Especially, the rampancy of antibiotics-resistant bacteria is causing a lot of concerns. In this example, it was investigated whether the skin gene card and the genetic test according to the present invention can be of help in diagnosis of the difficult-to-diagnose tuberculoderm. From a patient diagnosed of tuberculoderm by biopsy, skin lesion sample was acquired using the skin gene card of the present invention. The sample was added to a centrifuge tube and treated with 4% NaOH. Then, after adding sterilized distilled water to a total volume of 50 mL, centrifuge was performed at 3,000 rpm for 20 minutes. After discarding the supernatant and adding Tris EDTA (10 mM Tris-HCl [pH 8.0], 1 mM EDTA) buffer, centrifuge was performed at 7,000 rpm for 5 minutes. This procedure was repeated 2 times. After completely removing the supernatant, the precipitate was dissolved in 50-200 μL of 5% Chelex 100 and Tris EDTA buffer. After boiling the mixture for 10 minutes, centrifuge was performed at 12,000 rpm for 5 minutes. The supernatant (1-2 μL) was subjected to PCR. The supernatant (2 μL) was added to a reaction mixture (18 μL) of 10 mM Tris-HCl (pH 8.0), 50 mM KCl, 1.5 mM MgC12, 400 μM dNTPs, 20 pM primer sets and 2.5 U Taq DNA polymerase. After mixing well, PCR was performed under the following conditions.

TABLE 9
Mycobacterium tuberculosis DNA PCR primer

| Conventional nested | | |
|---|---|---|
| PCR | Type | Sequence (5′->3′) |
| First step PCR | Outer F | ATCCGCTGCCAGTCGTCTTCC |
| | (F-1) | |
| | Outer R | CTCGCGAGTCTAGGCCAGCAT |
| | (R-1) | |
| Second step PCR | Inner F | CATTGTGCAAGGTGAACTGAGC |
| | (F-2) | |
| | Inner R | AGCATCGAGTCGATCGCGGAA |
| | (R-2) | |
| Internal standard | HBB-F | GGCAGACTTCTCCTCAGGAGTC |
| for comparison | HBB-R | CTTAGACCTCACCCTGTGGAGC |

TABLE 10
Mycobacterium tuberculosis DNA PCR condition

| PCR condition | First step PCR | Second step PCR |
|---|---|---|
| Initial denaturation | 96° C./3 min | 96° C./3 min |
| Cycles | 35 cycles | 25 cycles |
| Denaturation | 95° C./30 sec | 96° C./3 min |
| Annealing | 60° C./30 sec | 96° C./3 min |
| Elongation | 72° C./1 min | 96° C./3 min |
| Final elongation | 72° C./10 min | 96° C./3 min |
| PCR product | 239 bp | 194 bp |

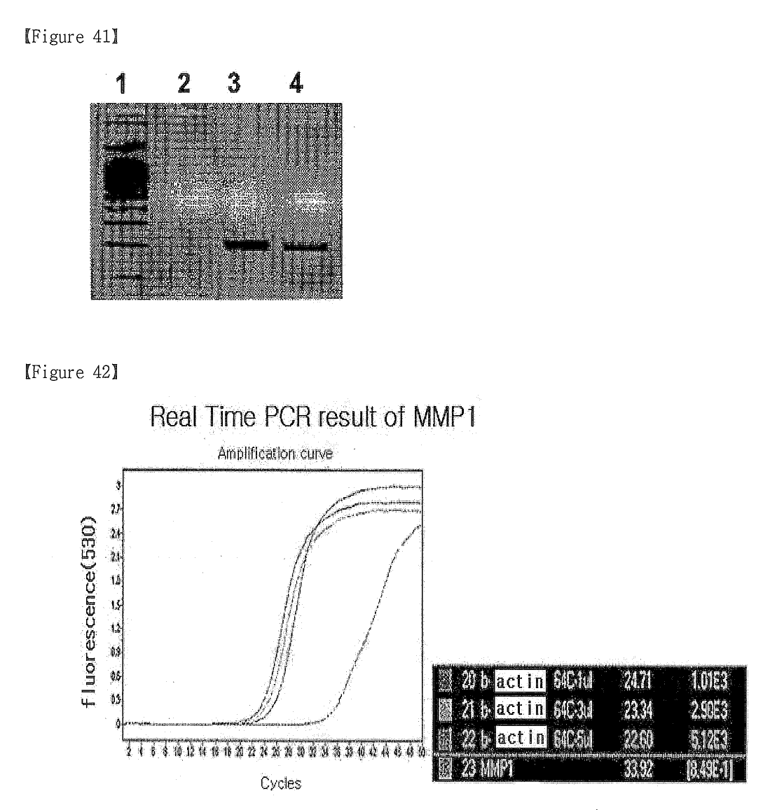

Following the second step PCR, electrophoresis was carried out on 2% agarose gel at 90 V for 40 minutes. Then, the gel plate was placed on a transilluminator. The result was evaluated as positive when 285 bp DNA fragment was observed. A 100 bp DNA ladder was used as DNA size marker, and DNA extracted from Mycobacterium tuberculosis separated from clinical sample served as positive control. In order reduce cross-contamination, the procedure of DNA extraction from the sample and the procedure of amplification were separated from each other [FIG. 41].

### Example 28

### Diagnosis by Test of Expression of Skin Condition- and Health-Related Genes Using Sample Acquired from Skin Gene Card

Candidate genes supposed to be of help in determining and classifying skin condition and determining personalized skin care were selected from the genes reported to be expressed normally or pathologically in the skin. Through a preliminary test, 31 genes (8 groups) playing important roles in synthesis and degradation of skin matrix proteins, lipid metabolism, melanogenesis, moisturization, proliferation and regeneration of skin cells, damage repair, differentiation, death, or the like and involved in skin aging, photoaging, regeneration, skin whitening, elasticity, moisturization, oiliness, immunity, inflammation, or the like were selected. RT-PCR and real-time PCRs for these genes and the house-keeping gene β-actin were established. The base sequences of primers adequate for real-time PCR of the genes and reaction conditions are given in Table 11.

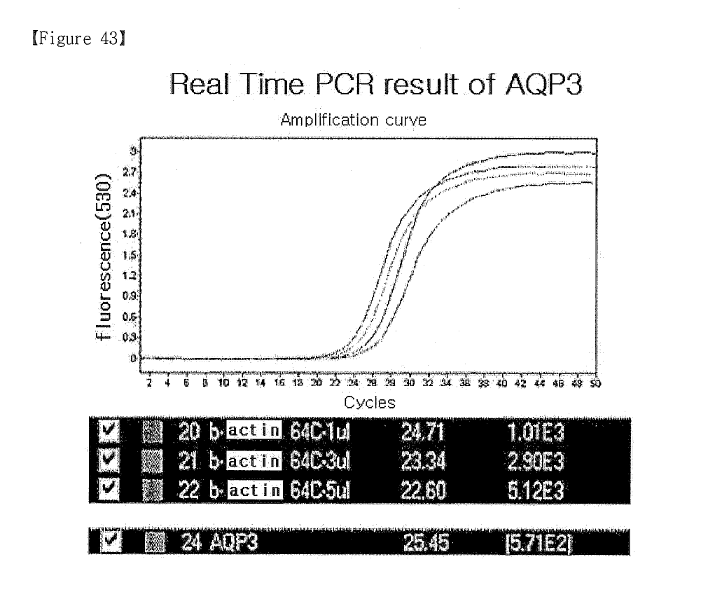

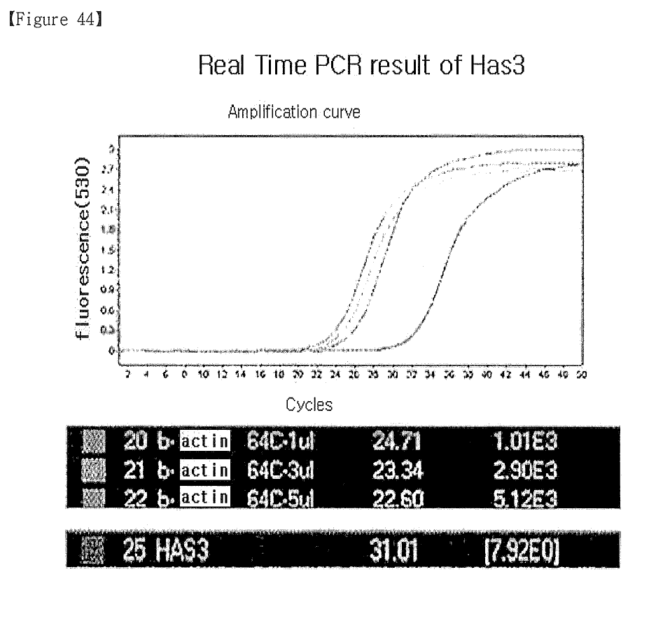

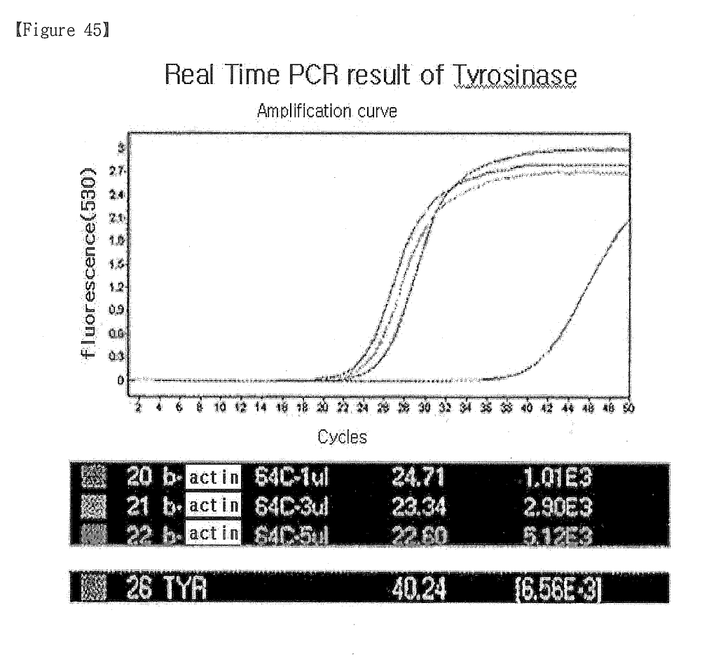

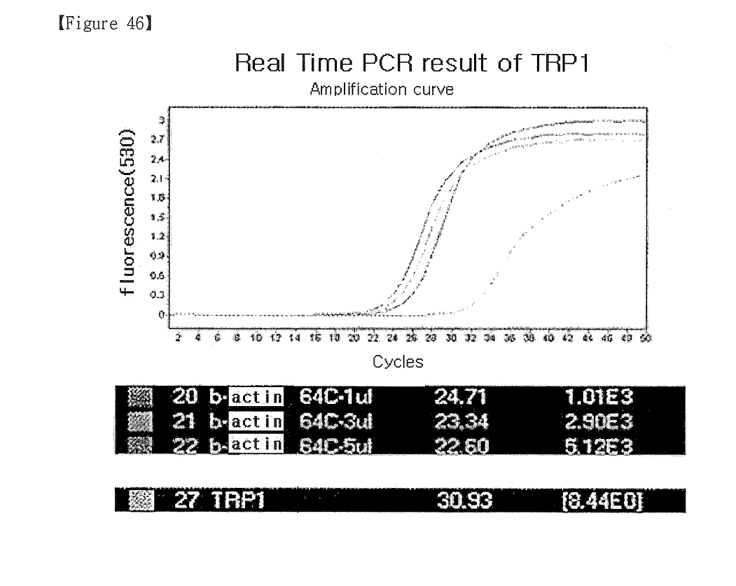

As specific examples, real-time PCR experiments for MMP1, HAS3, AQP3, tyrosinase and TRP3 and the results thereof are described below (Real-time PCR conditions for the five genes were identical) [FIGS. 42-46].

TABLE 11

| | | | Product |
|---|---|---|---|
| | Sequence | Direction | size |
| MMP1 | 5-CCGGTTTTTCAAAGGGAATAA-3 | Forward | 103 bp |
| | 5-CACAGTTCTAGGGAAGCCAAAG-3 | Reverse | |
| COL1A1 | 5-GGCGGCCAGGGCTCCGACCC-3 | Forward | 119 bp |
| Collagen and | 5-AATTCCTGGTCTGGGGCACC-3 | Reverse | |
| procollagen | | | |
| Tissue inhibitor of | 5-AATTCCGACCTCGTCATCAG-3 | Forward | 115 bp |
| metalloproteinase | 5-GGCTTGGTAACCCTTTATACATCA-3 | Reverse | |
| Elastin | 5-GCATTTCCCCCGAAGCT-3 | Forward | 122 bp |
| | 5-CTAACCCAAACTGGGCGG-3 | Reverse | |
| Elastinase | 5-CAGACAGAACTGGGTGATGACA-3 | Forward | 122 bp |
| | 5-CAGTGCCATCATTCTGGCTC-3 | Reverse | |
| Elafin | 5-TGGCTCCTGCCCCATTATC-3 | Forward | 71 bp |
| | 5-CAGTATCTTTCAAGCAGCGGTTAG-3 | Reverse | |
| MnSOD | 5-GGTAGCACCAGCACTAGCAGC-3 | Forward | 228 bp |
| | 5-GTACTTCTCCTCGGTGACGTTC-3 | Reverse | |
| Glutathione | 5-CATCTCCCTCATCTACACCAACTATGA-3 | Forward | 134 bp |
| S-transferase | 5-GTCTTGCCTCCCTGGTTCTG-3 | Reverse | |
| P53 | 5-GGAGGGGCGATAAATACC-3 | Forward | 131 bp |
| | 5-AACTGTAACTCCTCAGGCAGGC-3 | Reverse | |
| Telomerase | 5-GGTTTTTGAGGGTGAGGGTGAGGGTGAGGGTGAGGGT-3 | Forward | 116 bp |
| | 5-TCCCGACTATCCCTATCCCTATCCCTATCCCTATCCCTA-3 | Reverse | |
| Has3 | 5-CGATTCGGTGGACTACATCCA-3 | Forward | 142 bp |
| | 5-GTCGTACTTGTTGAGGATCTGGAC-3 | Reverse | |
| AQP3 | 5-CTTGCCCAAATAGCACCTTAGG-3 | Forward | 109 bp |
| | 5-ACATACCTGCTGCCCATTCTC-3 | Reverse | |
| Profilaggrin | 5-GGACAACTACAGGCAGTCTTGAAGA-3 | Forward | 126 bp |
| | 5-CATTTGCATGAAGACTTCAGCG-3 | Reverse | |
| TYR | 5-GGTTCCATGGATAAAGCTG-3 | Forward | 139 bp |
| | 5-GGGACATTGTTCCATTCATA-3 | Reverse | |
| TRP1 | GTCTTCACAATTTGGCTCAT-3 | Forward | 109 bp |
| | 5-AAGACTGCATCTGTGAAGGT-3 | Reverse | |
| ET-1 | 5-GCCCTCCAGAGAGCGTTATG-3 | Forward | 116 bp |
| Endothelin | 5-AGACAGGCCCCGAAGGTCT-3 | Reverse | |
| IL-1a | 5-GGCCCTGCATCACTTCAT-3 | Forward | 74 bp |
| | 5-TTGTTGAGGACAGCGACAATG-3 | Reverse | |
| TNF-a | 5-ACGCTCTTCTGTCTACTGAACTTCG-3 | Forward | 104 bp |
| | 5-ATAGCAAATCGGCTGACGGTGTGG-3 | Reverse | |
| CHL1(I-CAM) | 5-AGAACCTCAACCCACAATCA-3 | Forward | 281 bp |
| | 5-AAGCAAAGAACTCGCAATGT-3 | Reverse | |
| MHC2 | 5-AAGGATACCCAGATCCACC-3 | Forward | 105 bp |
| | 5-CTCAGCATTACGCTTTTGC-3 | Reverse | |
| HMG-CoA reductase | 5-TGGCTACGATGTCTCCCTACA-3 | Forward | 84 bp |
| | 5-CAACCCACACACCTGATGAA-3 | Reverse | |
| FAS | 5-AGCCTAACTCCTCGCTGCAAT-3 | Forward | 196 bp |
| (Fatty acid synthase) | 5-TCCTGGAACCGTCTGTGTTC-3 | Reverse | |
| Acc | 5-CTTTGGCAGGTTCCAGAG-3 | Forward | 129 bp |
| (Acetyl CoA carboxylase) | 5-TCCTCCAAATGTCATAGCC-3 | Reverse | |
| SPT-LCB1 | 5-CGAGGCTCCAGCATACCATC-3 | Forward | 132 bp |
| (Serine palmitoyl | 5-TGGCTGCCACTCTTCAATCA-3 | Reverse | |
| transferase) | | | |
| TGF | 5-CGAAGCTCAACTCAACTGTTTCTAC-3 | Forward | 108 bp |
| | 5-GCATTGGCATTCATGTTGGCAT-3 | Reverse | |
| Epithelial growth | 5-CAATACCGTTAAGATACAGTGTAGGC-3 | Forward | 95 bp |
| factor | 5-ACATTTTTCATTGATTCATCACAAC-3 | Reverse | |
| Keratinocyte growth | 5-CGCAAATGGATACTGACACG-3 | Forward | 148 bp |
| factor | 5-GGGCTGGAACAGGTTCACACT-3 | Reverse | |
| VEGF | 5-TAGTGATTCTGCCCTCCTCCTTC-3 | Forward | 215 bp |
| | 5-TGATGATTCTGCCCTCCTCCTTC-3 | Reverse | |
| 5 a reductase | 5-GAGCTTTTCACCACCATAGGTTCT-3 | Forward | 135 bp |
| | 5-AGTTTTCATCAGCATTGTGGGAGC-3 | Reverse | |
| Androgen receptor | 5-GCCCATTGACTATTACTTTCCAC-3 | Forward | 144 bp |
| | 5-CACAGGTACTTCTGTTTCCCTTC-3 | Reverse | |
| β-actin | 5-CTTCCAGAGTTTGTACTAGACCCAGT-3 | Forward | 146 bp |
| | 5-CCCTGCTGTACCTCTTTTAGACC-3 | Reverse | |

28-1. One-Step RT-PCR and RT-PCR of RNA Acquired from Skin Gene Card

Light Cycler ver 3.5 (Roche) and RT-PCR kit (Cyber Green Cat # 204243, Qiagen) were used and the supernatant (8 ul) was used as template.

The primers of the target genes listed in Table 1 were used as primers.

Cyber Green kit (Cat # 204243) was used for real-time PCR.

1) RT-PCR

a. Acquired RNA (1 ug) is added to a.

b. Oligo dT (100 pmol, 1 ul) is added.

c. The microtube is kept at 95° C. for 5 minutes.

d. The microtube is put on ice for 5 minutes.

e. After adding 10 mM dNTP, Expand RTase (Roche, 20 units), 5×RT buffer (3 ul) and RNase inhibitor (5 units), H20 is added to a final volume of 30 ul.

f. The microtube is kept at 43° C. for 1 hour, and then at 95° C. for 5 minutes.

g. The microtube is stored at 4° C. (cDNA synthesis completed).

2) Real-Time PCR

a. Light Cycler condition is set as follows.

reverse transcription: 50° C./20 min.

Predenaturation: 94° C./5 min.

Amplification: 94° C./15 sec, 55° C./20 sec, 72° C./20 sec.

Melt curve analysis: 95° C./5 sec, 64° C./15 sec, 95° C./0 sec.

Cooling: 40° C./30 sec.

b. A reaction solution for real-time PCR is prepared by mixing the followings.

Cyber Green mix 10 μL

RT mix 0.2 μL

Primer 1 μL

Template 4, 6, 8 μL

DEPC water 4.8 μL (to a final volume of 20 ul).

c. Control GAPDH is prepared by mixing the followings.

Template 4 μL, 6 μL, 8 μL

DEPC H20 4.8 μL, 2.8 μL, 0.8 μL

Primer 1 ul

Cyber Green mixture reaction solution 10.2 ul (to a final volume of 20 ul).

Cyber Green mixture reaction solution: Cyber Green mix (185 ul)+RT mix (3.7 μL)

d. Sample is loaded on a capillary and the cap is closed.

e. The capillary is quickly spun down on table top.

f. The capillary is mounted on Light Cycler and run is started.

### Example 29

### Establishment of Guideline for Personalized Skin Care Based on Test of Expression of Skin Condition- and Health-Related Genes Using Sample Acquired from Skin Gene Card

The purpose of this example is to apply the genetic test method established in Example 28 to skin care, beauty care and cosmetology. Above all, the inventors aimed at establishing a system capable of classifying skin type more accurately and objectively and being of help in selecting personalized skin care, cosmetics and cosmeceuticals. Especially, focus was made on accurately detecting dry, sensitive, naturally aged and photoaged skin, and providing an accurate diagnosis and treatment. To this end, study was made on 150 Korean women aged between 18 and 50 years. All of the subjects had visited beauty clinics or dermatological clinics and had their skin type determined through medical examinations by interview, physical examinations, or examinations using various instruments. All of them volunteered for this genetic test. Among them, 140 people had their skin type determined using Aphrodite skin diagnosis system (PSI, Seoul, Korea). The skin diagnosis system measures the skin's oil condition, water content, thickness of the horny layer, size of skin pores and depth of wrinkles, and estimates oiliness, dryness and agedness of the skin. Of the 150 subjects, 78 (52.0%) were evaluated as normal skin, 24 (16.0%) as dry skin, 16 (10.7%) as oily skin, and 32 (21.7%) as mixed type. Among them, 12 were determined to have severely sensitive skin, and 19 showed distinct skin aging. 22 had a lot of melasma.

The normal skin refers to a condition without skin disease and with no special discomfort. In the normal skin, cornification, loss of corneocytes, moisturization, and secretion of sebum and sweat are well balanced. The skin texture is soft and shiny, the skin surface is smooth and elastic, the pores are small, and the skin color is clear. A regular basic care for balancing oil and water content is sufficient for the normal skin.

In the dry skin, the water content in the stratum corneum is low. When measured with a corneomoter, an abnormally low water holding capacity of the stratum corneum is measured. And, when measured with an evaporimeter, an abnormally increased transepidermal water loss is observed. The skin surface is rough and scale develops. The skin is easily damaged by slight stimulations. The skin texture is soft but inelastic. Since the skin is thin, it ages and is lost easily, and shows sensitive reactions. After face wash, there is a sense of stretching. The skin is itchy and making up is difficult. In winter, the condition becomes severer. The dry skin tends to develop into sensitive or aged skin.

The oily skin is glossy and rough and pores are enlarged. Especially, the so-called T-zone, including forehead, nose and chin, is distinct. The oily skin is frequently accompanied by acne and enlarged capillary vessels, and develops well in young age after puberty.

The mixed type skin refers to a combination of two or more skin types. There are many cases where the T-zone (forehead, nose and chin) is oily and the U-zone (cheek and eye rims) is normal or dry. Acne and comedo develop well on the forehead, and wrinkles are formed well around the eye rims. Adverse reactions may occur when the same makeup is applied to various regions. The mixed type skin is common in the elderly.

The sensitive skin shows sensitive responses to seasonal or temperature changes, environmental changes such as stresses and UV, and contacts to cosmetics, soaps or other substances. Itching, flare and inflammation occur frequently, and pigmentation and enlargement of capillary vessels are frequently accompanied. (Sung-ku Ahn, Seung-Hun Lee. Skin aesthetics. Korea Medical Book Publisher. 2002).

The skin aging may be classified into natural, intrinsic or chronological aging, genetic aging, solar or photoaging, aging caused by lifestyles, endocrine aging, aging caused by chronic consumptive disease, aging caused by gravity, or the like (Pierrrd G E. Ageing across the life span: time to think again. Journal of Cosmetic Dermatology. 3:50-53; 2004). Among them, intrinsic aging and photoaging are the most common and important. The two are different in mechanisms, symptoms and signs. The intrinsic aging is characterized by smooth skin texture, fine and thin wrinkles, thin epidermis, normal or decreased elastic fiber, slightly decreased capillary vessels, and positive tumors, if any. In contrast, photoaging develops as the whole skin structure is damaged by sunlight, particularly UV, and it is recovered abnormally. It is characterized by rough and thick skin, rough and deep wrinkles, solar elastosis wherein the Grenze zone occurs in the papillary dermis as the elastic fibers become abnormally thick, significantly reduced but enlarged capillary vessels (resulting in skin redness), and not infrequent precancerous lesion, which may develop into malignant tumors (Korea Medical Book Publisher. 2002; Korean Dermatological Association Textbook Publishing Committee. Dermatology. 4th Edition. Ryo Moon Gak. 2001).

Skin sample was taken from the face, cheek and eye rims of the subjects using the skin gene card of the present invention. For RNA acquired from the sample, real-time RT-PCR for the 30 skin-related key genes in Example 28 and the house-keeping gene β-actin. Thereafter, the difference of expression of each target gene between skin types was statistically analyzed. It was investigated whether the expression of a specific gene significantly increased or decreased in a specific skin type as compared to the normal skin. The expression of target genes was measured as a ratio relative to that of the β-actin gene. The result was evaluated as meaningful when the value was significantly higher or lower than that of the normal skin group. In that case, the overexpression or underexpression of the specific gene can be viewed as related with the specific skin type and, and thus may be a standard for determining the skin types. The inventors tried to find a combination of target genes for the diagnosis of each skin type. For example, the sensitive skin exhibits significantly increased expression of immunity- and inflammation-related genes such as interleukin-1 alpha, tumor necrosis factor alpha, intercellular adhesion molecule-1 (1-CAM1), etc. as compared to the normal skin. Therefore, for those who have sensitive skin, a skin care method capable of preventing overexpression of cytokines and inflammation has to be provided. Use of irritant cosmetics or cosmeceuticals has to be avoided, and a patch test for hypersensitiveness may be required before applying cosmetics. As another example, in the case of severe skin aging, the expression of MMP-1 increases and that of TIMP, procollagen-1, procollagen-3, superoxide dismutase (SOD), epidermal growth factor (EGF) and keratnocyte growth factor (KGF) decreases. In that case, the skin care needs to be focused on inhibiting MMP1 and supplementing collagen, growth factors and antioxidatives.

There is a case not belonging to any of the above skin types. For instance, change in the expression of melanin-related genes may lead to pigmentation such as melasma. In that case, cosmetics and cosmeceuticals are selected focusing on inhibition of the expression of those genes.

The inventors also tried to find genes closely related with age by investigating correlations between the expression of each target gene and the age of the subjects. As a result, they identified that the expression of MMP-1 is in direct proportion to age. Hence, the test of the expression of MMP-1 gene may be a useful tool for predicting age.

The related examples are described in more detail in the followings.

However, the described genetic tests are not perfect by themselves and it is important to combine with other test results. Further, there may be a variety of variations and combinations depending on cases and portions of the skin. Besides, since the skin type may be incessantly changing, comparison test is required before and after skin care. Further, sustained skin care through follow-up is important. In addition to skin care, maintenance of the health of the whole body, adequate diet and nutrition, good lifestyle and mental health are important in keeping the skin healthy and beautiful.

### Example 29-1

### Application of Genetic Test and Personalized Skin Care for Oily Skin

In the oily skin group, the expression of HMG CoA reductase, fatty acid synthase, acetyl CoA carboxylase and serine palmitoyl transferase (SPT) genes, which play critical roles in lipid synthesis in the stratum corneum and sebum, and androgen receptor gene increased significantly as compared to the normal skin. This result indicates that the increased expression of these five genes is closely related with oily skin. Hence, when such phenomenon is observed in the genetic test, the skin may be evaluated as oily skin. The oily skin is cared focusing on the removal of excessive sebum. However, it is important to strike a balance, because disruption of the epidermal lipid may result in the breakdown of the epidermal barrier, thereby resulting in dry skin. It is important to use cosmeceuticals from mild one to more powerful ones.

(Step 1) Cleansing:

Face is washed using soap, cleansing lotion and gel. A foaming soap containing salicylic acid is adequate.

(Step 2) Toner:

After face wash, witch hazel astringent and alcohol-rich skin lotion are applied to the T-zone (forehead, eye rims and nose) to remove sebum.

(Step 3) Moisturizer:

Moisturizer including vitamin B3 (niacinamide) or natural vitamin A (retinol) is used to prevent skin dryness.

(Step 4) Removal of Sebum:

Cream including a polymer component capable of binding to and removing the remaining sebum is used. The cream is applied 1-3 times a week, and oils and wastes are removed by massage.

(Step 5) Sebum-Controlling Cosmetics

Talcum powder type cosmetics are used to remove the remaining sebum.

(Step 6) Vitamins

If excessive sebum secretion continues or severe acne develops, synthetic vitamin A compounds (retinoids) are administered.

### Example 29-2

### Application of Genetic Test and Personalized Skin Care for Dry Skin

In the dry skin group, the expression of hyaluronate synthase-3 (HAS-3), which synthesizes the powerful water-containing substance hyaluronic acid (hyaluronan) in the epidermis, aquaporin-3 (AQP3) gene, which is a water channel protein, profillaggrin gene, which is a precursor of natural moisturizing factor (NMF) in the epidermis, HMG CoA reductase and fatty acid synthase, which are lipid synthases, acetyl CoA carboxylase, serine palmitoyl transferase (SPT) gene, and procollagen-1 gene decreased significantly as compared to the normal skin group. In addition, the expression of MMP-1 gene increased. This result indicates that the change of the expression of the genes is closely related with skin dryness. When the skin lacks moisture because NMFs are not produced in the skin, or because the epidermal barrier is damaged due to abnormality in lipid metabolism or collagen synthesis, the skin becomes dry. Further, skin dryness is closely related with the skin barrier damage and aging. The fact that a variety of skin barrier damage-related diseases, e.g. eczema, psoriasis, icthyosis, atopic dermatitis occur frequently in dry skin and that the dry skin is frequently found in aged people and diabetic patients may be due to this mechanism. The skin exhibiting such an expression profile may be evaluated as dry skin. The care of dry skin is focused on solving the fundamental cause, i.e. supplying moisture to the stratum corneum and keeping it moist and strengthening the skin barrier in order to prevent water loss. In addition, focus is placed on relieving the itching or burning sensation. Details are as follows.

1) Soap and body cleanser containing mild surfactant are used for face wash. Excessive cleansing is avoided. The use of soap is reduced. Clothes giving severe frictions are abstained from.

2) The number of bath and shower is reduced. The indoor temperature is kept low and adequate humidity is maintained.

3) After face wash, emollient lotion with low alcohol content is used.

4) Oil-rich nourishing lotion is used.

5) After face wash, cream containing vitamins A, C and E, essence and oil are used for moisturization.

6) Fine wrinkles are treated by regularly using eye cream or essence.

7) Pack or massage is employed regularly 1-2 times a week.

8) Vitamin A-rich food is recommended.

9) Moisturizer or skin humectant capable of supplying and keeping moisture is used. Natural moisturizing substances, e.g. amino acid, urea, pyrrolidone carboxylic acid (PCA) and sodium lactate, panthenol, which is of help in skin moisturization and recovery of skin barrier, polyol substances, e.g. glycerol and glycerine, or polymer moisturizers, e.g. hyaluronic acid, chondroitin sulfate, collagen, etc. are used.

10) Occlusive agent which forms an impermeable layer on the skin surface to prevent water loss is used. Vaselines, lanolin, jojoba oil, cocoa butter, olive oil, dimethicone, cyclomethicone, and fatty acid complexes are adequate.

11) Oil-in-water or water-in-oil type skin emollient which smoothens and softens the skin surface is used. A mixture of cetyl stearate, dicaprylyl maleate, C12-C15 alkyl benzoate, etc. may be used.

12) Lipid capable of replacing the lipid existing in the stratum corneum of the epidermis is administered to recover the skin barrier. In this case, it is important to prepare the lipid into a natural lipid mixture, as in the natural stratum corneum, by mixing ceramide, cholesterol and free fatty acid equimolarly or intensifying ceramide and cholesterol.

13) It is recommended to avoid use of skin drugs. In case of severe itching or secondary change on the skin, adrenocortical hormones or antihistamines may be used.

### Example 29-3

### Application of Genetic Test and Personalized Skin Care for Mixed Type Skin

All the mixed type skin cases were a combination of oily skin at the T-zone and normal or dry skin at the U-zone. In the T-zone, the expression of HMG CoA reductase, fatty acid synthase, acetyl CoA carboxylase, SPT and androgen receptor genes was significantly increased as compared to the normal skin group, as in Example 29-1. And, in the U-zone, the expression of HAS-3, AQP3, profillaggrin, HMG CoA reductase, fatty acid synthase, acetyl CoA carboxylase, SPT and procollagen-1 genes was significantly decreased as compared to the normal skin group. The oily portion may be cared as in Example 29-1, and the dry portion may be cared as in Example 29-2.

### Example 29-4

### Application of Genetic Test and Personalized Skin Care for Sensitive Skin

In the sensitive skin, the expression of immunity- and inflammation-related genes such as interleukin-1 alpha (IL1 α), tumor necrosis factor alpha (TNF α), I-CAM, etc. was significantly increased as compared to the normal skin group. Further, as in the dry skin, the expression of MMP1 gene was increased and the expression of profillaggrin, HMG CoA reductase, fatty acid synthase, acetyl CoA carboxylase, SPT and procollagen-1 genes was significantly decreased as compared to the normal skin group. This may be because the sensitive skin is caused primarily by the increased expression of cytokines in the corneocytes of the epidermis due to external or intrinsic stimulation, which facilitates migration and activation of immune cells and induces inflammation. This suggests that the sensitive skin may be accompanied by skin barrier damage and skin dryness. Overexpression of IL-1 α or TNFα gene may be evaluated as sensitive skin. The care of the sensitive skin is focused on resolving the fundamental cause, i.e. reducing stimulations and inhibiting immunity and inflammation. Details are as follows.

1) Lifestyles and environments need to be changed. Skin-irritating substances or environments should be avoided. Abrupt temperature change such as hot bath or fomentation, as well as excessive abrasion or long-time bath, is to be avoided. Exposure to sunlight needs to be avoided if possible. Intake of pungent and hot food needs to be reduced. Environments need to be improved, such as pets, fur mats, ticks, etc. Mental stress needs to be relieved.

2) Less irritant cleanser or lotion is used. During face wash, weakly alkaline soap is used to reduce stimulation.

3) Adequate oil and moisture are provided using moisturizing lotion or cream.

4) Use of irritant cosmetics or cosmeceuticals should be avoided. A patch test for hypersensitiveness may be required before applying cosmetics.

5) The most recommendable cosmetics components are allantoin and bisabolol, which are not irritant to the skin and reduce inflammation, and panthenol, which is effective in skin moisturization and skin barrier recovery. In case of severe inflammation or if the expression level of IL-1 α and TNF α genes is higher than that of β-actin, cosmetics containing green tea extract may be used.

6) When exposed to sunlight, strong sunscreen cream or lotion is applied.

### Example 29-5

### Application of Genetic Test and Personalized Skin Care for Aged Skin

In the aged skin, especially photoaged skin, changes in expression of various genes was observed. First, the expression of MMP-1, which degrades collagen, the main protein component of the dermis, was significantly reduced. And, the expression of procollagen-1, procollagen-3 and TIMP increased, thereby resulting in fiber loss of the dermis. Second, the expression of HMG CoA reductase, fatty acid synthase, acetyl CoA carboxylase and SPT, which are involved in the production of the lipid of the skin barrier, and profillaggrin, the precursor of natural moisturizing factor, decreased, thereby resulting in skin barrier loss. Third, the expression of superoxide dismutase, an important enzyme removing superoxide radicals and preventing damage occurring therefrom, was decreased. Fourth, the expression of growth factors required for skin regeneration, i.e. epidermal growth factor (EGF), keratnocyte growth factor (KGF) and basic fibroblast growth factor (basic FGF), and vascular endothelial growth factor (VEGF) was significantly decreased. This indicates that the shortage of growth factors and improper tissue regeneration may be the important cause of aging. Besides, the photoaged skin group exhibited increased expression of elafin and decreased expression of elastase. This result indicates that skin aging is caused by the change in expression of the aforesaid genes, and results from the general structural and functional insufficiency of the skin tissue. Such changes in gene expression may be diagnosed as skin aging, without regard to age. The care of the aged skin is focused on resolving the fundamental cause. Details are as follows.

1) Cosmetics containing antioxidative substances are used. Oral administration may be of help. The antioxidative substances include plant-derived substances and vitamins. The former includes polyphenol extracted from green tea, quercetin, genistein, pyncogenol, ellagic acid, or the like, and the latter includes vitamin E, vitamin C, vitamin A, alpha lipoic acid, ubiqinone, idebenone, etc.

2) Cosmetics containing growth factors (EGF, KGF, bFGF) may be used.

3) Cosmetics containing MMP1-inhibiting components or collagen may be used. For example, the pentapeptide Pal-KTTKS, collagen-1 fragment, polyphenol extracted from green tea, quercetin, nobilin, neovastat, may be used.

4) Lipid capable of replacing the lipid existing in the stratum corneum of the epidermis may be used. In particular, a natural lipid mixture prepared by mixing ceramide, cholesterol and free fatty acid equimolarly or intensifying ceramide and cholesterol may be used.

### Example 29-6

### Application to Genetic Test and Personalized Skin Care for Melanin-Related Gene Expression Anomaly

Of the 150 subjects, 22 showed distinct melasma. They exhibited significantly increased expression of tyrosinase, TRP1 and endothelin-1 (ET1) genes as compared to the normal skin group. Among them, tyrosinase and TRP1 are key genes involved in melanogenesis, and ET-1 is a cytokine conjectured to regulate the proliferation of melanocytes. The result indicates that the overexpression of the three genes is closely related with excessive pigmentation. Hence, the overexpression of the genes may be evaluated as high risk of pigmentation.

The high risk group is treated by administering hydroquinone, azelaic acid, kojic acid, glabridin, aloesin, vitamin A or vitamin B3 (nicianamide), which inhibit the activity or action of tyrosinase. A possible consideration is administering 4% hydroquinone together with vitamin A, and then applying a moisturizer containing azelaic acid, kojic acid and glabridin.

### Example 29-7

### Application to Skin Age Determination and Skin Care

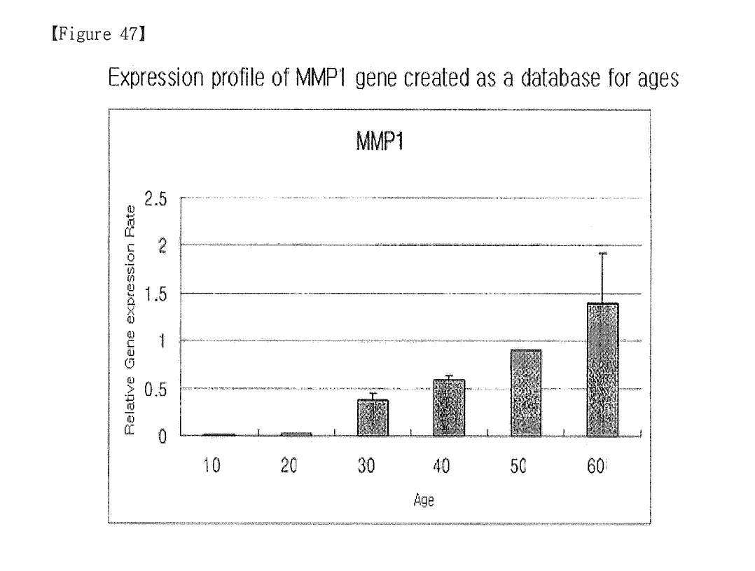

Of the 150 subjects, 58 showed distinct photoaging with age. This group showed a proportional increase in the expression of MMP1 [FIG. 47]. In general, it is known that the expression of MMP1 increases as photoaging proceeds. In particular, at the age around 40, when aging begins, the expression starts to increase abruptly.

It is known that skin aging begins at the age around 25. But, it progresses fully at around 40 years. As skin ages, it becomes dry due to decreased excretion, cell regeneration is slowed, and the skin becomes rough due to the accumulation of aged horny layer. Further, wrinkles are formed due to decreased collagen synthesis and denaturation of elastin. In addition, the skin is discolored and pigmentation occurs such as melasma and dark spots. The epidermis becomes thinner and provides less skin protection. Besides, skin troubles increase due to the decrease of skin thickness and skin barrier action. These physiological actions may be diagnosed by determining the expression level of aging-related genes.

Among many skin aging types, the most important two types are intrinsic aging and photoaging. Of the tested 150 subjects, 58 showed progressing skin aging. They exhibited shorter telomeres as compared to the normal group, and 10-1,000 times more expression of MMP1 than β-actin (endogenous control). This result suggests that the telomere length and the overexpression of the MMP1 gene are closely related with skin aging. Especially, the overexpression of MMP1 gene, particularly that 0.001 or more than the expression of β-actin, may be diagnosed as risky.

FIG. 47 shows the expression profile of MMP1 gene created as a database for ages, for predicting skin age of a subject. For example, if the relative expression of MMP1 is 1×10⁻³, the subject's skin age may be predicted as early 30s.

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A skin gene card comprising:
a tape portion for acquiring tissue from a human body by attaching and detaching it to and from the human body; and
a card portion for protecting, storing and transporting the acquired tissue.

2. The skin gene card according to claim 1, wherein the tape portion is a low-tack paper bandage.

3. The skin gene card according to claim 1, wherein the card portion comprises a material selected from a group consisting of paper card, glass slide, OHP film, plastic, polyester, fiber, metal and combinations thereof.

4. The skin gene card according to claim 3, wherein the card portion comprises a paper card, wherein the paper card is prepared by sterilizing the paper card using an autoclave, followed by immersion in cell lysis buffer, uric acid and/or chitosan treated with diethylpyrocarbonate (DEPC), and drying.

5. The skin gene card according to claim 4, wherein the paper card is immersed in a water-soluable chitosan solution with a concentration ranging from 0.02% (w/v) to 0.25% (w/v).

6. The skin gene card according to claim 1, wherein the tissue from the human body is hair or mucosa taken at the skin-mucosa interface.

7. A method for acquiring human body tissue using a skin gene card, comprising:
attaching the tape portion of the skin gene card according to claim 1 on the skin at the sampling portion of the human body; and
detaching the tape portion from the skin.

8. The method for acquiring human body tissue using a skin gene card according to claim 7, further comprising, prior to said attaching, removing horny substance on and around the skin at the sampling portion using a peeling gel.

9. The method for acquiring human body tissue using a skin gene card according to claim 7, wherein the tape portion attached to the skin of the human body is detached 1 minute to 12 hours after the attaching the tape portion to the skin.

10. A method for separating nucleic acids from human body tissue, comprising:
acquiring human body tissue using the skin gene card according to claim 1; and
separating nucleic acids using a nucleic extraction means.

11. The method of claim 10, further comprising:
(PCR) or reverse transcription (RT)-PCR amplification of the separated nucleic acids without further purification of separated nucleic acids.

12-13. (canceled)

14. The method of claim 10, further comprising
performing multiplex PCR amplification of short tandem repeat (STR) polymorphisms in the separated nucleic acids; and
identifying an individual from genetic information obtained from the nucleic acids amplified by the multiplex PCR.

15. A method for pharmacogenomic testing using a skin gene card, comprising:
acquiring human body tissue using the skin gene card according to claim 1 and separating genomic DNA from the acquired tissue;
performing multiplex PCR of a drug metabolism-related gene selected using the separated genomic DNA; and
identifying genetic information amplified by the PCR.

16. A kit for nutrigenomic testing or diagnosis of disease comprising:
the skin gene card according to claim 1; and
forward and reverse primers targeting a disease-related gene, or forward and reverse primers for multiplex PCR of a gene selected from the group consisting of obesity-, antioxidative stress-, detoxification-, cardiovascular disease-, hormone metabolism-, allergy- and bone metabolism-related genes.

17. (canceled)

18. The kit for diagnosis of disease according to claim 16, wherein the disease is a skin cancer and the gene is a melanoma antibody.

19. The kit for diagnosis of disease according to claim 16, wherein the disease is a skin infectious disease and the gene is a pathogen-specific gene.

20. The kit for diagnosis of disease according to claim 19, wherein the pathogen is Staphylococcus aureus.

21. The kit for diagnosis of disease according to claim 16, wherein the disease is a sexually transmitted disease and the gene is a pathogen-specific gene.

22. The kit for diagnosis of disease according to claim 21, wherein the pathogen is selected from a group consisting of N. gonorrhea, C. trachomatis, M, genitalum, M. hominis, U. urealyticum and T. vaginalis.

23. The kit for diagnosis of disease according to claim 21, wherein the pathogen is selected from a group consisting of H. ducreyi, G. vaginalis, T. pallidum, Herpes simplex virus, Candida albicans and human papillomavirus (HPV).

24. The kit for diagnosis of disease according to claim 17, wherein the pathogen is a Tubercle bacillus-specific gene.

25. A method for skin gene expression test using a skin gene card, comprising:
acquiring human body tissue using the skin gene card according to claim 1 and separating genomic RNA from the acquired tissue;
performing reverse-transcription PCR of a skin condition- and health-related gene selected using the separated genomic RNA; and
identifying the expression level of the gene amplified by PCR.

26. The method for skin gene expression test according to claim 25, wherein the gene is a gene selected from a group consisting of matrix metalloproteinase 1 (MMP1), procollagen A1, tissue inhibitor of metalloproteinase (TIMP), elastin, elastase, elafin, superoxide dismutase 1 (MnSOD, SOD1), glutathione S-transferase, p53, telomerase, hyaluronan synthases 3 (HAS3), aquaporin 3 (AQP3), profillaggrin, tyrosinase, tyrosinase-related protein 1 (TRP-1), endothelin-1, tumor necrosis factor-α (TNF-α), I-CAM, major histocompatibility complex 2 (MHC2), 3-hydroxy-3-methylglutaryl-coenzyme A (HMG-CoA), fatty acid synthase, acetyl CoA carboxylase, transforming growth factor-β1, epidermal growth factor (EGF), keratinocyte growth factor (KGF), vascular endothelial growth factor (VEGF), 5-α reductase and androgen receptor, or a house-keeping gene.

27. The method for skin gene expression test according to claim 25, wherein the expression level of the gene is identified by real-time PCR.
